(19) United States
(12) Patent Application Publication
(10) Pub. No.: US 2019/0223936 A1
(43) Pub. Date: Jul. 25, 2019

(54) INDICATING SYSTEM AND METHOD FOR ELECTROSURGICAL INSTRUMENT

(71) Applicants: Matthew Otten, Cincinnati, OH (US); Gary L. Long, Cincinnati, OH (US); Jeffrey A. Benz, Indianapolis, IN (US); Richard L. Grant, Cincinnati, OH (US)

(72) Inventors: Matthew Otten, Cincinnati, OH (US); Gary L. Long, Cincinnati, OH (US); Jeffrey A. Benz, Indianapolis, IN (US); Richard L. Grant, Cincinnati, OH (US)

(73) Assignee: SECQURE SURGICAL PTE. LTD., Singapore (SG)

(21) Appl. No.: 16/336,799

(22) PCT Filed: Sep. 26, 2017
(86) PCT No.: PCT/US2017/053568

§ 371 Date: Mar. 26, 2019

Related U.S. Application Data
(60) Provisional application No. 62/400,053, filed on Sep. 26, 2016.

(51) Int. Cl. A61B 18/12; A61B 18/14

(52) U.S. Cl. CPC A61B 18/1206; A61B 2018/00898; A61B 2018/1226; A61B 18/1445

(57) ABSTRACT

Systems and associated methods for sensing and indicating when a wide range of tissue is adequately cauterized and/or sealed by an electrosurgical instrument. The system indirectly monitors the current flowing through the tissue and determines the adequacy of tissue cauterization or sealing of vessel(s) by monitoring when the current is stable or nearly stable (i.e. when the current is constant). The system may also indicate a predetermined time that current is applied through the tissue and control the flow of energy through the tissue.

### CROSS REFERENCE TO RELATED APPLICATIONS

This application claims priority to U.S. Provisional Patent Application No. 62/400,053, filed on Sep. 26, 2016, entitled “INDICATING SYSTEM AND METHOD FOR ELECTROSURGICAL INSTRUMENT.” The entire disclosure of the foregoing provisional patent application is incorporated by reference herein.

### BACKGROUND

The present invention relates to electrosurgery devices and systems, as well as methods for performing electrosurgical procedures. More particularly, the present disclosure relates to indirect current monitoring for electrosurgical devices and systems, as well as systems and methods for determining the state (e.g., the adequacy) of electrosurgical treatment of tissue based on a rate of change of current, and/or determining the length of time that electrosurgical energy has been applied to tissue via indirect current monitoring.

Electrosurgery generally involves the application of high frequency (i.e., radio frequency, or “RF”) current (also referred to as electrosurgical energy) in order to seal, cauterize, and/or coagulate tissue as a result of heat generated within the tissue. Electrosurgical devices may also be used to cut, ablate (fulgurate), and/or desiccate tissue. RF current, when applied to tissue, causes an increase in intracellular temperature. In some electrosurgery applications, tissue is heated in a controlled manor such that small blood vessels are sealed, blood is coagulated, and other tissue is cauterized. Sealing is achieved by heat bonding (coagulating) the proteins in the tissue. For sealing larger blood vessels or other lumens, pressure is applied in combination with RF current.

RF current is alternating current having a frequency within the radio frequency portion of the electromagnetic spectrum. As an alternating current, RF current periodically reverses its direction of flow with the voltage polarity periodically reversing. When used in electrosurgery, RF current may be continuous or pulsed, with various waveforms employed (e.g., sinusoidal, square, triangular, etc.). RF current (i.e., the level, magnitude or amplitude of the current), since it alternates in direction, is often determined as the root mean square, or RMS, over one (or more) cycles. As is known to those skilled in the art, RF current can also be quantified in a variety of other ways that take into account the fact that it alternates in direction, such as the peak (or crest) value, the peak-to-peak value, ½ of the peak-to-peak value, or the average value (i.e., the average of the absolute values) of the waveform over one cycle. Unless the context indicates otherwise, as used herein “RF current” refers to the amplitude (i.e., magnitude) of the current as determined by the RMS, peak, peak-to-peak, ½ peak-to-peak, average or other measure of the magnitude of an alternating current. Similarly, unless the context indicates otherwise, as used herein the “voltage” of an alternating signal (e.g., a voltage alternating in polarity, or an alternating voltage signal on a DC bias) refers to the amplitude (i.e., magnitude) of the voltage as determined by the RMS, peak, peak-to-peak, ½ peak-to-peak, average or other measure of the magnitude of an alternating voltage signal.

RF current for electrosurgery is typically supplied and controlled by an electrosurgical generator (often referred to as an Electrosurgical Unit, or ESU) or other source of RF current, with leads or cables running between the generator and a hand-held electrosurgical instrument. ESUs used in operating rooms generally convert current at standard electrical frequencies supplied from a wall outlet, which are typically 50 or 60 Hz (depending on location), to much higher frequencies—e.g., from about 350 to about 800 kHz, with some commercially available ESUs operating as high as 4000 kHz.

There are two basic electrosurgery techniques for completing an electrical circuit for delivering the electrosurgical energy to tissue: monopolar and bipolar. In monopolar electrosurgery, an active electrode is used to apply the electrosurgical energy to the targeted tissue to achieve the desired surgical effect. RF current passes from the active electrode to the targeted tissue, and then through the patient to a remotely positioned grounding pad (also referred to as a return electrode), and then back to the generator to complete the circuit. The grounding pad (or return electrode) is typically positioned beneath the patient, in direct contact with the patient's skin. The active electrode is provided by the hand-held instrument, such as at the distal end of an end effector located on or mounted to the instrument.

In bipolar electrosurgery, both the active and return electrodes are provided by the instrument, such as at the distal end of an end effector located on or mounted to the instrument. One or more electrodes of the instrument functions as the active electrode and another as the return electrode, with the return electrode located in close proximity to the active electrode(s). The targeted tissue is positioned between the active and return electrodes (e.g., between the jaws of bipolar electrosurgical forceps), and RF current is passed from the active electrode to the return electrode through the targeted tissue. In this manner, the delivery of electrosurgical energy is targeted to the tissue positioned between the electrodes.

Compression of the tissue during electrosurgical treatment can be necessary for adequate vessel sealing and hemostasis. Bipolar open electrosurgical forceps as well as bipolar endoscopic electrosurgical forceps utilize both mechanical clamping action and electrical energy to effect hemostasis and sealing. In some instances, a cutting blade is also provided in order to cut the tissue after sealing. Typically, a blade slot is provided on one or both electrodes, and the cutting blade is driven through the slot in order to cut the tissue through the center of the sealed tissue region.

The active and return electrodes of bipolar electrosurgical forceps are typically provided on opposing jaw members that can be selectively closed in order to clamp tissue between the jaw members, and opened in order to separate the electrodes and release the sealed (and, in some instances, cut) tissue. When the opposing jaw members are in spaced-apart relationship, the electrodes are sufficiently separated from one another such that the electrical circuit is open and current will not flow between the active and return electrodes even if there is inadvertent contact between the electrodes and body tissue. When the jaw members are closed and grasp tissue, RF current can be selectively delivered through the tissue. A surgeon can cauterize, coagulate, desiccate and/or simply reduce or slow bleeding, by controlling the intensity, frequency and duration of the RF energy applied between the electrodes and through the tissue. Overly desiccating the target tissue is often undesirable because surrounding tissue may be damaged from residual heat, the tissue may stick to the electrodes making it difficult to remove the electrosurgical instrument from the tissue without tearing or damage, and/or adequate structural integrity of an electrosurgically sealed blood vessel or other lumen may be lost.

Electrosurgical systems generally include an electrosurgical instrument coupled to an energy source (e.g., an ESU). The ESU provides, and oftentimes controls the electrosurgical energy delivered to the tissue for treatment purposes. Many ESUs are controlled by hand operated switches and/or other forms of input devices provided on the handheld electrosurgical instrument itself and/or on a foot switch connected to the ESU and operable to, for example, start and stop the delivery of RF current to the active electrode(s). RF generators also typically include manual controls for setting predetermined parameters (e.g., power level and/or wave form selection) for particular applications (e.g., tissue cutting and/or coagulation).

ESUs, in addition to providing a source of electrosurgical energy, often are configured to control the delivery of RF current based on predetermined parameters such as a predetermined tissue impedance level. Predetermined impedance levels are almost always empirically developed for a specific tissue treatment modality and/or a specific electrode (or device) configuration. ESUs attempt to directly measure the tissue impedance (Z) using an additional electrode provided on the electrosurgical instrument, or calculate (i.e., estimate) the tissue impedance by measuring the output current (when the generator is delivering energy having a constant voltage) or voltage (when the generator is delivering energy having a constant current). However, since the size (e.g., the diameter of the tissue lumen to be sealed), type and other characteristics of the targeted tissues vary greatly, it is difficult to develop suitable predetermined parameters that work well over a wide range of tissues. As a result, the usable range of the ESU is limited or the predetermined parameter features (e.g., shutting off power when tissue impedance reaches a certain level) do not work well for over a wide range of tissues. Also, such systems generally require a matched pair of a generator and a handheld electrosurgical instrument in order to determine tissue impedance and/or to match that impedance to a predetermined impedance level. The impedance-based control schemes of these generators typically will not work if, for example, one manufacturer's generator is used with another manufacturer's electrosurgical forceps.

The use of various sensing devices and circuitry has been proposed in the prior art for purposes of employing various predetermined algorithms for applying RF current to tissue. However, such sensing arrangements have not been widely adopted. Sensors have been proposed for determining various tissue properties, including temperature, real and/or imaginary impedance, conductivity, transmittance, opacity, and the like. Suitable RF current properties have also been used in conjunction with one or more of these tissue properties, including voltage, current, power, energy, and phase. Non-limiting examples of sensors suitable for measuring tissue and/or energy properties include thermal sensors, electromagnetic field sensors, impedance monitors, optical sensors, transformers, capacitive sensors, and various combinations of the foregoing. However, none is believed to provide a simple, low cost and compact indicating or sensing system for indicating, for example, when tissue has been adequately sealed and/or cauterized.

### BRIEF DESCRIPTION OF THE DRAWINGS

While the specification concludes with claims particularly pointing out and distinctly claiming the invention, it is believed that the invention will be better understood from the detailed description of certain embodiments thereof when read in conjunction with the accompanying drawings. Unless the context indicates otherwise, like numerals are used in the drawings to identify similar elements in the drawings. In addition, some of the figures have been simplified by the omission of certain elements in order to more clearly show other elements. Such omissions are not necessarily indicative of the presence or absence of particular elements in any of the exemplary embodiments, except as may be explicitly stated in the corresponding detailed description.

FIG. 1 is an annotated graphical representation of an electrosurgical RF current curve with respect to time.

FIG. 2 is a graphical representation of the first time derivative of the RF current in FIG. 1.

FIG. 3 is a schematic illustration of one embodiment of an electrosurgical system having an indicating system located between an energy source and an electrosurgical instrument operatively connected to the energy source.

FIG. 4 schematically depicts a more detailed illustration of an embodiment of the electrosurgical system of FIG. 3, wherein the indicating system is located between an RF generator and a handheld electrosurgical forceps instrument.

FIG. 5 schematically depicts a more detailed illustration of an embodiment of the electrosurgical system of FIG. 3, wherein the indicating system is integral to or contained within or on (in whole or in part) the housing of a handheld electrosurgical forceps instrument.

FIG. 6 provides a more detailed schematic illustration of an embodiment of the indicating system used in the systems depicted in FIGS. 4 and 5.

FIG. 6A provides a detailed block diagram of an embodiment of an indicating system particularly configured for being operatively connected between an RF generator and an electrosurgical instrument such as electrosurgical forceps.

FIG. 6B provides a detailed block diagram of an alternative embodiment of an indicating system particularly configured for being incorporated into an electrosurgical instrument such as electrosurgical forceps.

FIG. 7 depicts an embodiment of an electrosurgical system similar to that of FIG. 5, wherein the indicating system of FIG. 6B is incorporated into the housing of a handheld, bipolar electrosurgical forceps instrument.

FIG. 8 depicts a perspective view of the bipolar forceps of FIG. 7, wherein the length of the cable has been shortened for purposes of clarity.

FIG. 9 depicts a top view of the electrosurgical instrument of FIG. 8.

FIG. 10 depicts a side view of the electrosurgical instrument of FIG. 8 with the left handle half removed to show the interior of the electrosurgical instrument handle.

FIG. 11 depicts a cross-sectional view of the electrosurgical instrument of FIG. 8.

FIG. 12 is an exploded view of the elongated portion of the bipolar forceps electrosurgical instrument of FIG. 8, exposing the knife assembly.

FIG. 13 depicts a perspective view of an alternative embodiment of the end effector of a bipolar forceps electrosurgical instrument, wherein the end effector is straight rather than curved.

FIG. 14 depicts a perspective view of the end effector of the bipolar forceps electrosurgical instrument of FIG. 8.

FIG. 15 depicts a cross-sectional schematic view of an end effector portion of one embodiment of an electrosurgical instrument with the jaw members open and the knife retracted.

FIG. 16 depicts a cross-sectional schematic view of the end effector portion of FIG. 15 with the jaws in the clamped position and the knife advanced.

FIG. 17 is an exploded view of the elongated portion and end effort of the bipolar forceps electrosurgical instrument of FIG. 8.

FIG. 18 depicts a perspective view of the elongated portion of the bipolar forceps electrosurgical instrument of FIG. 8, with an exploded view of the driving assembly for driving the knife.

FIG. 19 depicts a perspective view of the end effector of the bipolar forceps electrosurgical instrument of FIG. 8, with an exploded view of one of the jaw members.

FIG. 20 depicts a perspective view of the bipolar forceps electrosurgical instrument of FIG. 8 with the left handle half removed to show the interior of the instrument handle.

FIG. 21 is an exploded view of the bipolar forceps electrosurgical instrument of FIG. 8, exposing the components of the instrument assembly.

FIG. 22 depicts a perspective view of the trigger of the bipolar forceps electrosurgical instrument of FIG. 8.

FIG. 23 depicts a perspective view of the safety member of the bipolar forceps electrosurgical instrument of FIG. 8.

FIGS. 24 and 25 depict front and rear views, respectively a standalone indicating system of FIG. 6A incorporated into a housing and configured for being operatively positioned between a generator and an electrosurgical instrument.

The drawings are intended to illustrate rather than limit the scope of the present invention. Embodiments of the present invention may be carried out in ways not necessarily depicted in the drawings. Thus, the drawings are intended to merely aid in the explanation of the invention. Thus, the present invention is not limited to the precise arrangements shown in the drawings.

### DETAILED DESCRIPTION

The following detailed description describes examples of embodiments of the invention solely for the purpose of enabling one of ordinary skill in the relevant art to make and use the invention. As such, the detailed description and illustration of these embodiments are purely illustrative in nature and are in no way intended to limit the scope of the invention, or its protection, in any manner. It should also be understood that the drawings are not to scale and in certain instances details have been omitted, which are not necessary for an understanding of the present invention.

As used herein, unless the context indicates otherwise, the term “cable” is intended to encompass signal-conducting devices comprising an assembly of two or more conductors such as wires (single or multiple strand), and other types of physical conduits, traces or lines that conduct electrical signals, whether power signals (e.g., RF current) or communication signals (e.g., a voltage or current indicative of a sensed condition, a video, image or audio signal, etc.). As also used herein, the phrase “in electrical communication” means that the electrical signals can be transmitted between the two components, such as via one or more wires, conduits, traces, lines, terminal blocks, posts, solder joints, integrated circuit traces, connectors, plugs and the like, or through direct contact of the two components.

Embodiments of the present disclosure provide systems and methods for determining the state (e.g., the adequacy) of an electrosurgical treatment of tissue based on the current flowing through the tissue (e.g., a rate of change of the current) and/or a determination of the length of time that current has been applied to the tissue. Embodiments are particularly useful with electrosurgical generators delivering energy at a constant (or nearly constant) voltage (including those delivering pulsed electrosurgical energy, wherein those pulses have a constant voltage). In some embodiments, current flowing through the tissue is indirectly monitored using magnetoresistive sensing. These embodiments employ, for example, a giant magnetoresistance (“GMR”) sensor to monitor RF current indirectly. The RF current does not flow through the GMR sensor. Instead, the GMR sensor is located to be within the magnetic field that surrounds the RF current-carrying conductor (e.g., a trace, wire or other conduit carrying the RF current) when RF current is being transmitted through that conductor. Also, while a GMR sensor can be used to determine the current (i.e., magnitude of the current), embodiments described herein utilize a GMR sensor to monitor the rate of change of current and/or to monitor the stages of tissue treatment. For example, the rate of change in RF current is used in controlling tissue treatment (e.g., controlling operation of the electrosurgical instrument and/or generator) and/or for determining (and, in some instances, indicating to the user) a state of tissue treatment (e.g., that adequate vessel sealing or tissue cauterization has been achieved). Thus, in some embodiments there is no need to precisely calibrate the sensing circuitry so as to be able to determine the actual level of RF current—only the rate of change of RF current is used. Also, in some embodiments it is not necessary to measure the voltage or other parameters of the electrosurgical energy delivered to tissue, nor is it necessary to calculate tissue impedance or other tissue or electrosurgical energy properties.

Furthermore, in some embodiments employing a GMR sensor to monitor current there is no need for circuitry to be in direct electrical communication with the generator or even the electrosurgical instrument itself in order to determine the onset of current delivery to tissue or the rate of change of that current. The GMR sensor only needs to be located immediately adjacent one of the two current paths, since it only indirectly monitors current. For this reason, embodiments described herein can be used with both bipolar and monopolar electrosurgery. In addition, the systems described herein can be configured to be inexpensive to manufacture as well as sufficiently compact to fit within a handheld electrosurgical instrument or mechanically coupled in an unobtrusive manner between a generator and a handheld electrosurgical instrument.

In some embodiments, a system and method for determining the adequacy of the electrosurgical treatment of tissue such as the sealing of a tissue lumen (e.g., a blood vessel) and/or cauterization of tissue during an electrosurgical procedure are provided, wherein the adequacy of the electrosurgical treatment is determined by monitoring the rate of change of current through the tissue and/or the length of time that current has been applied to the tissue. RF current flowing through the tissue is indirectly monitored in order to, for example, detect when the current is stable (or substantially stable), thereby indicating that adequate tissue treatment (e.g., tissue sealing) has been achieved.

In still further embodiments, the systems and methods described herein are employed to control an electrosurgical procedure. The electrosurgical procedure is controlled directly and/or indirectly. Direct control means that the delivery of RF current to the tissue is altered (e.g., ceased, current and/or voltage reduced, etc.) automatically based on a determination that the current through the tissue has a predetermined characteristic (e.g., when the current is stable). For example, when the rate of change of RF current through the tissue is substantially zero (i.e., the magnitude of RF current is substantially constant) for a predetermined period of time, indicating that adequate electrosurgical tissue treatment (e.g., vessel sealing) has been achieved, the delivery of electrosurgical energy to the tissue is automatically ceased. Such automatic cessation can be implemented by the generator, the instrument or by a separate device located between the generator and the instrument that automatically disrupts transmission of electrosurgical energy from the generator to the electrosurgical instrument or that signals the generator to cease delivery of electrosurgical energy.

Indirect control means that the indicating system provides an indication to the user (e.g., a surgeon) based on a determination that the current through the tissue has a predetermined characteristic (e.g., when the rate of change of current is substantially zero). The indication can be visual (e.g., one or more lights or other visible indicia), audible (e.g., a buzzer or other audible indicia) and/or tactile (e.g., vibration or other form of tactile feedback). The control is indirect in that the user decides what to do in response to the indication—e.g., causing the cessation of the delivery of electrosurgical energy to the tissue when the indication is provided, or shortly thereafter, such as by deactivating a hand or foot switch or other actuator for current delivery. In some instances, a plurality of sensed or determined parameters can trigger an indication to the user. For example, if an indication has been provided to the surgeon that vessel sealing is complete, but the surgeon has not caused the delivery of current to cease within a predetermined period of time after the first indication is provided, a second indication (e.g., a louder audible signal, indicator light begins flashing, etc.) is provided to the surgeon.

In still further embodiments, the electrosurgical procedure is controlled both indirectly and directly. For example, when current through the tissue has a first predetermined characteristic (e.g. when the rate of change of current has decreased to a predetermined level, such as substantially zero), an indication (visual, audible and/or tactile) is provided to the user. The user then determines whether to take some action (e.g., ceasing the delivery of electrosurgical energy to the tissue) in response to that indication. Direct control (e.g., cessation of electrosurgical energy delivery) is also provided whereby the delivery of electrosurgical energy to the tissue is controlled (e.g., ceased, current and/or voltage reduced, etc.) automatically when the current through the tissue has a second predetermined characteristic (different from the first predetermined characteristic) or when the user has not taken action within a predetermined period of time following delivery of the indication. For example, in one embodiment if an indication has been provided to the surgeon that vessel sealing is complete, but the surgeon has not caused the delivery of current to cease within a predetermined period of time after the first indication is provided, the delivery of RF current is automatically ceased (e.g., by the RF generator or by the instrument).

In some embodiments, the indicating system is incorporated into the RF generator itself, such that upon a determination of adequate tissue treatment, the RF generator stops supplying current (i.e., direct control) to the electrosurgical instrument (e.g., electrosurgical forceps) or provides an indication to the user (e.g., a visual and/or audible indication is provided by the generator). In other embodiments the indicating system is incorporated into the electrosurgical instrument (e.g., within the instrument housing) for monitoring current, and (a) regulating the delivery of that current (direct control, e.g., terminating the delivery of current to tissue upon a determination of adequate tissue treatment); or/and providing an indication to the user (e.g., a visual and/or audible indication is provided by the instrument).

In still further embodiments, the indicating system is located between the RF generator and the electrosurgical instrument (e.g., located along the cables connecting the generator and instrument). In these embodiments, the indicating system can be configured for use between one manufacturer's RF generator and another manufacturer's electrosurgical instrument, particularly when the indicating system is configured to monitor the rate of change of RF current rather than determine the amount of RF current. The indicating system of these embodiments can be in the form of a housing (e.g., a box) having suitable electrical connectors such that the cable that would normally extend between the RF generator and the electrosurgical instrument is operatively connected between a first set of electrical connectors on the indicating system and one of the generator and instrument, and a second cable is connected between a second set of electrical connectors on the indicating system and the other one of the generator and instrument. Thus, the indicating system in these embodiments is located in-line, between the RF generator (bipolar or monopolar) and the electrosurgical instrument.

In one particular embodiment the present disclosure provides systems and methods to indicate (or control tissue treatment in response to) a determination that tissue has been adequately cauterized and/or sealed by an electrosurgical instrument. The systems and methods can be used in the treatment of a wide variety of tissues (as to size, type, thickness, etc.). The system indirectly monitors the current flowing through the tissue in order to determine adequacy of tissue cauterization or vessel sealing. In some embodiments, the system determines when the rate of change in RF current flowing through the tissue decreases to a predetermined level, such as when the current is substantially stable (i.e., when the current stops increasing or decreasing for a predetermined period of time, such that the rate of change in RF current is approximately zero). Other embodiments detect when current is first applied to the tissue and compute the cumulative amount of time that current is applied. The system indicates to the user when the current is stable and/or when a predetermined period of time has elapsed since current delivery started so that the user can manually discontinue treatment (or the system can directly control the current by switching it off) or take other action(s) to decrease or discontinue the application of electrosurgical energy to the tissue.

In some embodiments, a giant magnetoresistance (GMR) type device is used to monitor RF current. GMR sensors are sensitive to small changes in a magnetic field, and are therefore capable of providing indirect sensing of current (as well as other electrical properties such as frequency or any other parameter that relates to electron spin physics). GMR devices utilize a quantum mechanical magnetoresistive effect observed in thin film structures composed of alternating ferromagnetic and nonmagnetic layers to change resistance due to the magnetic field being proportional to the current flowing in the conductive trace. A GMR sensor is typically constructed as two ferromagnetic metal films separated magnetically by a nonmagnetic film. GMR sensors are not placed in direct electrical contact with the conductor. Instead, the GMR sensor is placed in the magnetic field surrounding a current conductor (e.g., a wire lead or trace on a circuit board). The resistance of the GMR device changes in proportion to the strength of the magnetic field, and that magnetic field strength is proportional to the amount of current flowing through the adjacent conductor. Thus, the GMR sensor produces an output voltage that is proportional to the magnetic field, and hence the current creating that magnetic field. GMR sensors are typically fabricated as an integrated circuit (e.g., as a Small Outline Integrated Circuit, or SOIC) that incorporates the GMR sensor element and additional circuitry for providing an output voltage that is proportional to the magnetic field strength at the sensor element. GMR sensors are commercially available, for example, from NVE Corporation.

In embodiments further described herein, the GMR sensor (e.g., provided as part of an IC chip) is located within the magnetic field that surrounds one of the RF current conductors (e.g., a wire, a trace on a circuit board or other conductor) through which RF current travels either to or from one of the instrument electrodes and the targeted tissue. The GMR sensor thus provides a voltage signal that is proportional to, and therefore represents the RF current flowing through the targeted tissue—including the variability in the RF current over time. As the tissue is being treated (e.g., a blood vessel or other lumen is being sealed), the current through the tissue changes as the treatment progresses. This change in current results in a time-varying voltage signal from the GMR sensor. This changing GMR sensor output voltage is used to monitor the rate of change in RF current flowing through the tissue. The indicating system signals when the tissue is adequately sealed or cauterized based on that rate of change of the RF current, as represented by the rate of change in the sensor voltage signal, thereby allowing the cessation of the delivery of RF current to the tissue so that the tissue is not overly cauterized and avoiding excessive or destructive desiccation, charring, and/or sticking of the tissue to one or both electrodes. Because the systems and methods described herein are based on monitoring the rate of change of the RF current through the tissue rather than any measurement of the RF current itself, the rate of change of the time-varying voltage signal provided by the sensor is equivalent to the rate of change of the RF current, quantified as an arbitrary unit over time. Thus, when the RF current through the tissue is stable, the output voltage provided by the GMR sensor is also stable (e.g., the rate of change of the output voltage is within a predetermined range for a predetermined amount of time).

The amount of current needed to achieve adequate sealing or cauterization of blood vessels and other lumens will vary based on, for example, the size of the electrodes, the contact area of the electrodes, the amount of tissue between the electrodes, the pressure applied to the tissue, and tissue properties, including the tissue impedance or change in tissue impedance. When using electrosurgical forceps, for example, the time required for sealing and cauterization can vary from about 1 second for thin tissue or small vessels (e.g., 1 or 2 mm in diameter) to over 12 seconds for larger vessels (e.g., up to 7 mm in diameter or greater) or for very thick tissue. Even when adequately sealed, tissue impedance, and hence current, is not the same for all types and sizes of tissue being sealed, even when using the same forceps and generator.

Applicants have discovered, however, that for vessel/lumen sealing or cauterization using electrosurgical energy of a constant voltage, when the RF current flowing through targeted tissue is substantially stable, the tissue has been adequately sealed or cauterized—regardless of the type, properties or amount of tissue being sealed between the active and return electrodes. Thus, embodiments of the indicating systems described herein do not depend upon measuring or estimating tissue impedance or any other single property of the tissue, but instead determine when adequate sealing or cauterization is achieved based on the rate of change in current flowing through the tissue. This methodology can be applied regardless of the variables in tissue, electrode configuration, frequency, tissue density, electrical properties, RF energy waveform, etc., that complicate the efficacy or previous vessel sealing or tissue cauterization indicating or control systems.

One particular embodiment utilizes controller circuitry (also referred to herein as a “controller”) to determine adequacy of the seal or cauterization based on the indirect monitoring of RF current through the tissue using a GMR sensor. The controller circuitry causes one or more indicators to change state (e.g., to actuate) so as to notify the user, and/or the controller circuitry causes the cessation of RF current (e.g., by shutting down current from the generator). In one embodiment, the controller circuitry includes a differentiator circuit whose output voltage is directly proportional to the rate of change of the voltage signal from the GMR sensor. This output voltage is referred to herein as the “differential voltage” because it is proportional to the change in GMR sensor output voltage (V) with respect to time (t), or in calculus notation the differential dV/dt. The differential voltage is also proportional to the rate of change of the RF current through the tissue with respect to time, or dI/dt. In some embodiments, when the differential voltage from the comparator is less than a predetermined level, the indicating system triggers one or more indicators (light, buzzer, etc.) so as to alert the user that sealing or/and cauterization is complete. The control circuitry can have a variety of configurations, some of which are further described herein.

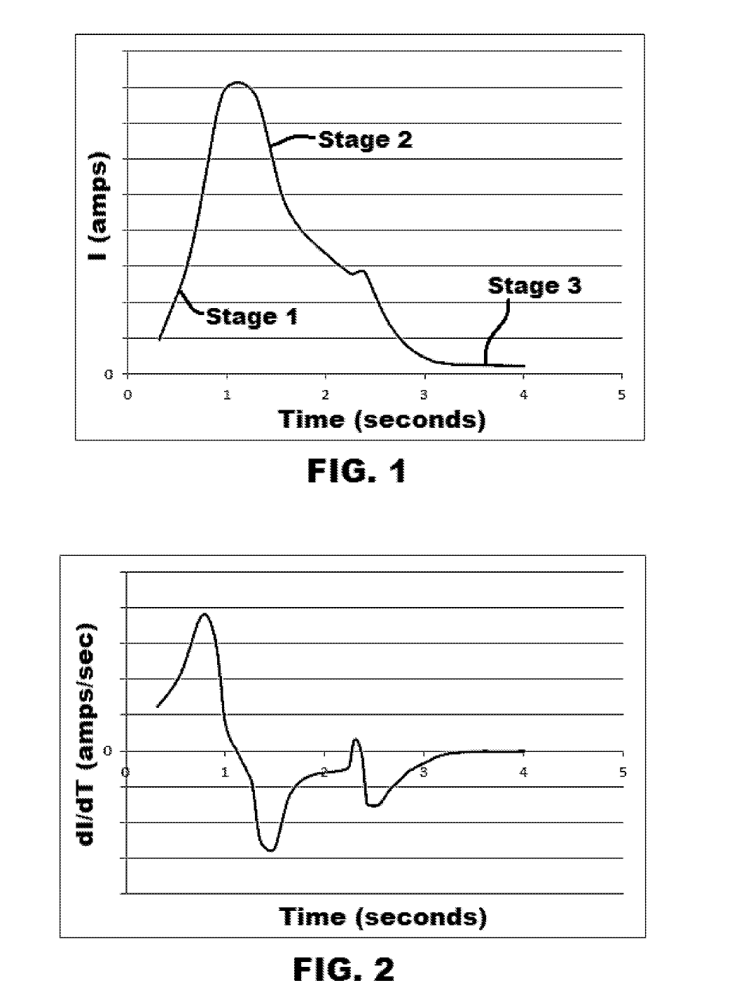

The controller circuitry can take a variety of different forms, and include a variety of components. In some embodiments the controller circuitry includes one or more processors such as a microprocessor (along with memory and I/O devices, either standalone or as part of a microcontroller having the microprocessor), an application specific integrated circuit(s) (ASIC), and/or a field programmable gate array(s). For example, some embodiments make extended use of a microprocessor programmed to determine the adequacy of the seal or cauterization based on the indirect monitoring of RF current, time of treatment and other data. An advantage of the use of a microprocessor (e.g., in the form of a microcontroller) is that it can also perform other functions such as: screening or filtering noise that is induced in the energy source (the ESU); compensating for variations introduced to the system by different energy sources (ESUs); compensating for system component tolerances within the controller circuitry; preventing premature indications from RF current fluctuations or anomalies; monitoring the progression throughout the sealing stages (see FIG. 1); controlling the indicators (visual, audible and/or tactile) that signal the device operator; and/or controlling ESU output.

Handheld electrosurgical instruments powered by external RF generators typically are connected to the generator via an electrical cable and connectors. However, this generally requires that the RF generator and electrosurgical instrument, cables and connectors are compatible because, for example, the generator employs a threshold impedance developed for a specific instrument(s) in order to control various functions. Therefore, in most instances an electrosurgical instrument from one manufacturer cannot be used with a generator from another manufacturer, and hospitals must purchase an ESU that is compatible with each brand or type of electrosurgical instrument its surgeons wish to use. Embodiments described herein, however, allow for the indicating system to be used between one manufacturer's RF generator and another manufacturer's electrosurgical instrument. In addition, the miniaturization of the sensing and control circuitry, along with a self-contained power supply in some instances, allows the indicating system to fit in a handheld electrosurgical instrument (or even within the cable or a connector for a handheld electrosurgical instrument). The indicating system can also be configured to be compatible with most ESUs and electrosurgical instruments.

In one embodiment, the indicating system comprises miniaturized circuitry that is on board the electrosurgical instrument to inform the user when sealing or cauterization is adequate. An on board power supply is included in the handheld instrument to power the GMR, microprocessor, indicators, and other electrical needs of the system and to eliminate the need for a separate external power cord or other power connection.

Other embodiments of systems and methods can be configured to monitor other characteristics of the current flowing through the tissue using a GMR sensor. For example, the system and methods can be configured to trigger indictor(s) upon a predetermined rate of change in current, impedance or other parameters such as power, waveform, voltage, pulse rate, etc. In some embodiments, the systems and methods of the present disclosure rely on the rate of change in RF current delivered to the tissue, which inherently takes into account tissue variables (thickness, fat/water content, etc.) without being dependent upon one or more predetermined tissue parameters to determine if tissue is adequately cauterized or vessel(s) are adequately sealed.

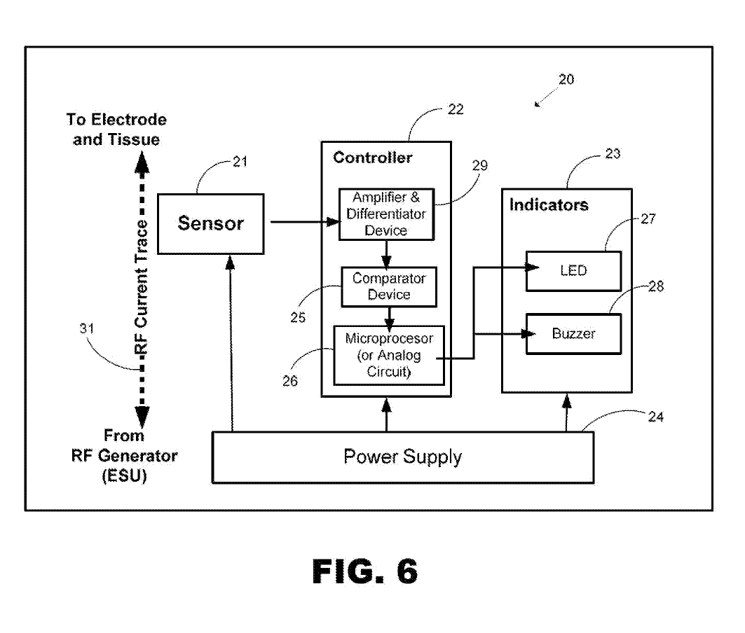

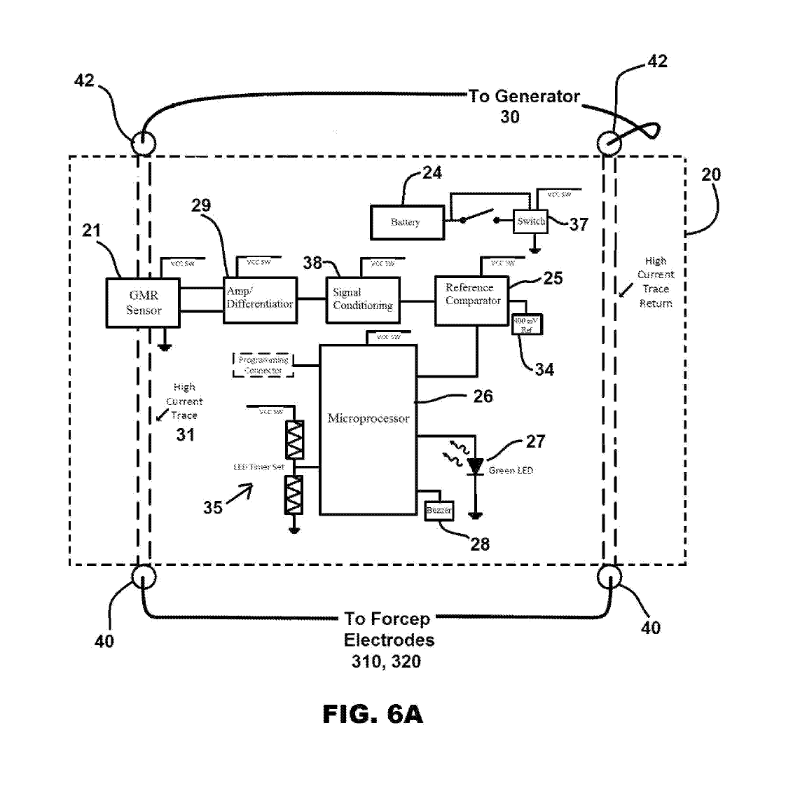

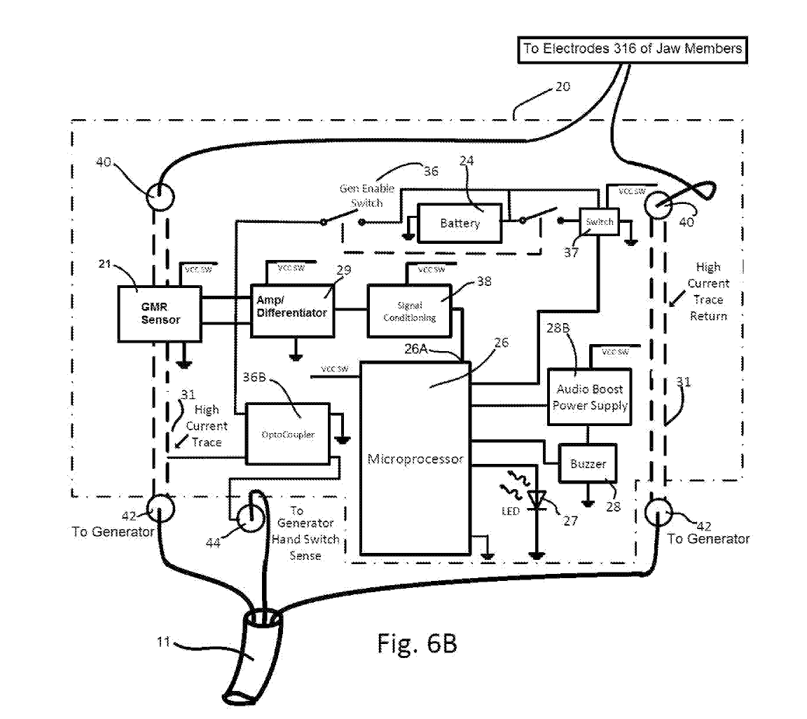

Another embodiment of an indicating system according to the present disclosure is useful in detecting an electrical short between the electrodes or within an electrosurgical instrument. In these embodiments, the indicating system is configured to indicate to the user that an electrical short has been detected based on the RF current being stable but at a higher than normal level. This detection allows the user to reposition the instrument on the tissue and continue treatment or take other corrective actions. The characteristics of an electrical short can be distinguished from normal sealing/cauterization based on the voltage output of the GMR sensor being stable, but at a level that is above a predetermined amount, thereby indicating the presence of an electrical short (i.e. a good indication of an electrical short is stable current at a higher than normal level). Therefore, in case of a short, the RF current can be switched off by the controller circuitry or the user can be notified by different signals to switch off the current. In the embodiment of FIG. 6B, for example, an additional signal can be provided to the microprocessor 26 wherein that signal is proportional to the magnitude of the RF current. Thus, when the microprocessor 26 determines that the rate of change of the voltage signal from the GMR sensor is stable (as further described herein), but the RF current is higher than expected, the microprocessor changes the state of an indicator so as to signal to the user that a short is likely.

Various embodiments described herein utilize an onboard power supply; provide simplicity and miniaturization of circuitry; and/or offer versatility in dealing with differing tissue and other variables for the indicating system to be incorporated in a disposable handheld component of a bipolar forceps vessel and tissue sealer or other electrosurgical instrument.

While specific embodiments will be described in connection with electrosurgical forceps and blood vessel sealing, in other embodiments the indicating system is used in conjunction with other types of electrosurgical instruments or treatments.

As mentioned previously, Applicants have discovered that blood vessels and other lumens have been adequately sealed, or tissue adequately cauterized, by electrosurgical techniques (e.g., using electrosurgical forceps) when the magnitude of the RF current through the tissue (resulting from the application of electrosurgical energy at a constant voltage) becomes constant with respect to time. A typical sealing cycle for one embodiment of the present disclosure is illustrated in FIG. 1. In this illustration, the RF current applied to the tissue is plotted with respect to time during a typical tissue cauterization or vessel sealing cycle, using an RF generator configured to apply a constant (or approximately constant) voltage between the treatment electrodes. As RF energy is applied to the tissue, the tissue impedance is low allowing the current level to increase, as identified as Stage 1 in FIG. 1. As the moisture in the tissue begins to dissipate, the impedance starts to increase and the current flow starts to drop, as identified as Stage 2 in FIG. 1. Once the tissue is adequately cauterized or the vessel adequately sealed, the current remains constant or nearly constant, as identified as Stage 3 in FIG. 1. Stage 3 may occur at any current level (or amperage), dependent upon, for example, the type of tissue being sealed or cauterized. If current is continued to be applied to the tissue after Stage 3 is reached, current continues to flow through the tissue (as seen in FIG. 1). The temperature of the tissue will continue to increase and moisture will continue to dissipate until the tissue is fully desiccated—a condition which exceeds the ideal tissue sealing or cauterization level. Overly desiccating the tissue is undesirable because surrounding tissue may be damaged and the targeted tissue will often stick to the electrode(s), making it difficult to remove the electrosurgical instrument from the tissue without tearing or damage.

FIG. 2 is a graphical representation of the first time derivative of RF current, showing that that the rate of change of current is approximately zero once sufficient vessel sealing or tissue cauterization has been achieved (i.e., Stage 3). It is this observation that forms the basis for some of the embodiments described herein. It will be understood that FIGS. 1 and 2 are merely exemplary, as, for example, the actual current through tissue depends on a number of variables including the tissue type and thickness, RF frequency, and even the brand of generator and forceps used.

Based on the discovery that adequate tissue lumen sealing or cauterization corresponds to a stabilization of the RF current, one embodiment of the present disclosure provides an indicating system having at least one sensor, controller circuitry, one or more indicators, and a power supply. The sensor is configured to sense one or more electrical parameters or properties during the application of electrosurgical energy to tissue, and is in electrical communication with the controller circuitry. The controller circuitry processes the signal(s) from the sensor so as to control activation of the indicator(s), and optionally control one or more other devices such as an ESU or other electrosurgical energy source. The sensor can be configured to detect or measure various electrical conditions such as, voltage, current, impedance, imaginary impedance, conductivity, power, energy, phase and other properties. In one particular embodiment, the sensor comprises a current sensor adapted for sensing the RF current flowing through an electrosurgical instrument, and hence RF current applied to tissue by one or more electrodes provided on the instrument.

FIG. 3 is a schematic illustration of one embodiment of an electrosurgical system comprising an energy source 30 (e.g., a bipolar Electrosurgical Unit or generator supplying current at a constant voltage) operatively connected to an electrosurgical instrument 10 (e.g., bipolar electrosurgical forceps) via one or more conductors 31, wherein tissue treatment is monitored by an indicating system 20. The indicating system 20 can be provided within the energy source 30, within the instrument 10, or between the energy source and the instrument, and provides an indication to the user when tissue treatment has been adequately completed. Upon such indication, the user can then manually control the operation of the instrument (e.g., using a button, a foot switch, or other actuating device to control operation of the electrosurgical instrument or the energy source) in response to such indication. A sensor of the indicating system 20 is located within the magnetic field surrounding an RF current conductor 31 (e.g., a trace on a circuit board or other electrical conductor through which RF current flows) and monitors the current indirectly (i.e., without direct contact or other electrical communication with the RF current conductor).

In the embodiment of FIG. 3, the user controls the delivery of electrosurgical energy to tissue in response to an indication from the indicating system 20 that tissue treatment is adequate. In alternative embodiments, feedback from the indicating system 20 to the energy source 30 and/or to the electrosurgical instrument 10 is provided (as shown by broken lines in FIG. 3) in order to control the delivery of electrosurgical energy to tissue and/or other functions of the energy source or instrument. Such feedback can be provided via one or more separate conductors, such as in an electrosurgical cable, or wirelessly (e.g. using Bluetooth technology).

The indicating system 20 can be constructed on a single circuit board that includes other functional components, as further described herein. In addition, in alternative embodiments the circuit board can include a remote switch or other device to control the RF energy source 30 via separate conductors in an electrosurgical cable or wirelessly (e.g. using Bluetooth technology). By way of example, the indicating system can include a switch or other mechanism that disrupts the electrical communication between the energy source 30 and the instrument 10, thereby causing cessation of the delivery of electrosurgical energy to tissue upon detection of a predetermined event (e.g., adequate tissue treatment, duration of tissue treatment, etc.). Circuit board manufacturing is low cost and compact, even allowing a handheld electrosurgical instrument 10 having the indicating system 30 incorporated therein to be disposable in some instances. Some embodiments of the indicating systems described herein also have the advantage of being compatible with a variety of bipolar generators or other energy sources.

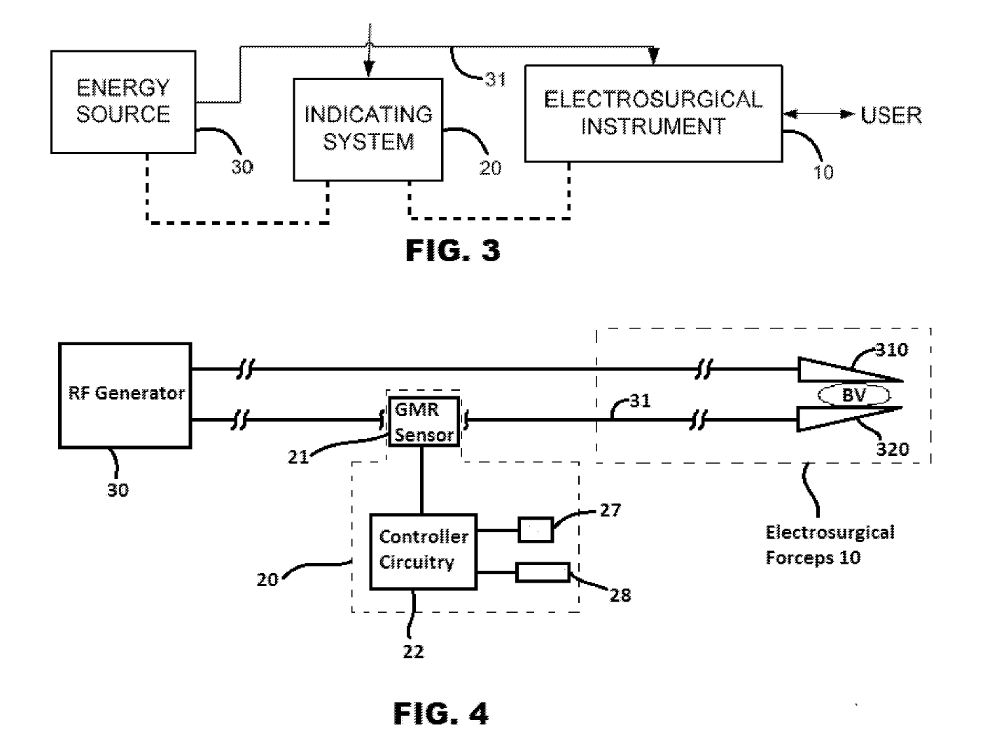

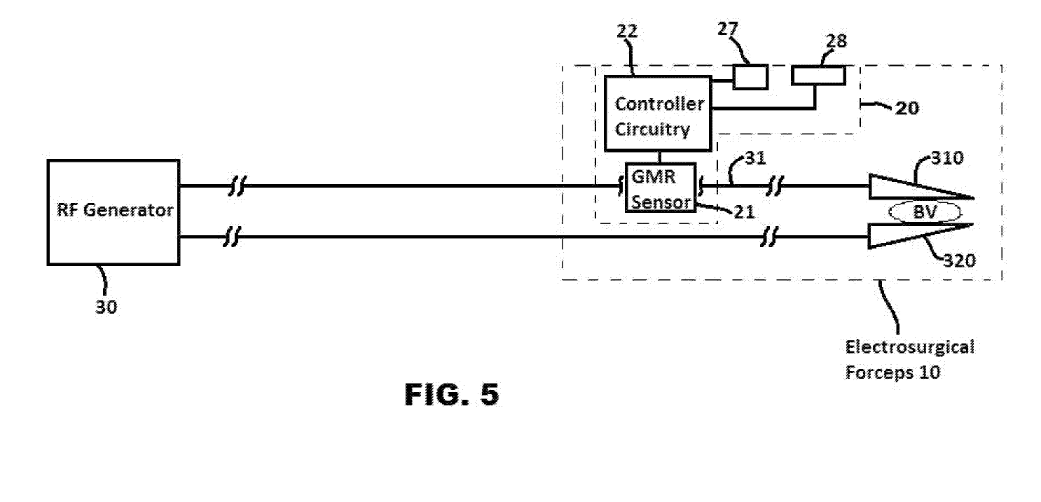

FIGS. 4 and 5 schematically depict more detailed illustrations of alternative implementations of the electrosurgical system of FIG. 3 comprising RF generator 30, electrosurgical instrument 10 and indicating system 20. In each of FIGS. 4 and 5, the indicating system 20 is used with (FIG. 4) or incorporated into (FIG. 5) electrosurgical forceps 10 having a pair of jaw members 310 and 320. Each of the jaw members 310, 320 has at least one electrode for applying current to tissue (e.g., a blood vessel, BV) clamped between the jaw members (i.e., bipolar forceps). It will be understood, however, that the indicating system 20 can be used with (or incorporated into) any of a variety of electrosurgical instruments, including monopolar forceps, electrocautery devices (e.g., electrocautery pencils), tissue ablation therapy devices, atrial appendage exclusion ablation devices, and microwave ablation devices (e.g., microwave ablation catheters).

In the electrosurgical system of FIG. 4, the indicating system 20 is located between the RF generator 30 and the electrosurgical instrument 10 (e.g., located along a cable connecting the generator and the instrument). In this embodiment, the indicating system 20 can be configured for use between one manufacturer's RF generator and another manufacturer's electrosurgical instrument. In the electrosurgical system of FIG. 5, the indicating system 20 is incorporated into the electrosurgical instrument 10. In both FIG. 4 and FIG. 5, the indicating system 20 generally comprises a GMR sensor 21, controller circuitry 22 for processing the signal(s) provided by the GMR sensor, one or more a visual indicators 27 (e.g., an LED) and one or more audible indicators 28 (e.g., a buzzer). The GMR sensor 21 is located adjacent one of the RF current-carrying conductors so as to be within the magnetic field that surrounds the conductor, whereby the GMR sensor provides a signal indicative of the RF current flowing through that conductor (e.g., a voltage signal that is proportional to the RF current). In the embodiment of FIG. 4, the GMR sensor 21 is located adjacent a RF current-carrying conductors that conducts RF current between the generator 30 and the instrument 10. In FIG. 5, the GMR sensor 21 is located adjacent a RF current-carrying conductor that delivers current to the active electrode 310 or a conductor that carries current from the return electrode 320. The controller circuitry 22 processes the signal provided by the GMR sensor and determines, for example, when the RF current has become constant for a predetermined period of time, thereby indicating adequate tissue treatment (e.g., vessel sealing or tissue cauterization). When the RF current has become constant, the indicating system will provide visual and audible signals to the surgeon (e.g., a visual indicator such as an LED will light and an audible indicator such as a buzzer will emit sound). The indicating system may also include other functionality, as further described herein.

FIG. 6 provides a more detailed schematic illustration of an indicating system 20, for use in the embodiments shown in FIGS. 4 and 5 as well as FIG. 6A. The indicating system 20 comprises a sensor 21, controller circuitry 22, one or more indicators 23 and a power supply 24. In this embodiment, the sensor 21 comprises a GMR sensor for sensing the RF current flowing through the electrosurgical instrument and tissue via an RF current conductor in the form of a trace 31, thereby generating a voltage signal that is proportional to the RF current. The RF current trace 31 provides electrical communication between the ESU and an end effector electrode of the instrument that delivers RF current through the tissue (e.g., for vessel sealing). The GMR sensor 21 is located within the magnetic field generated by the current flowing through the conductive RF current trace 31, and is positioned sufficiently close to the current trace to provide a useable voltage signal. A GMR sensor has the advantage of indirect sensing rather than requiring an electrical connection to the current trace in order to directly measure the current. The power supply 24 provides power to the GMR sensor 21, as well as to the controller circuitry 22 and indicators 23.

As seen in FIG. 6, the controller circuitry 22 is in electrical communication with the sensor 21 and the indicator(s) 23. An internal power supply 24 is provided such that the indicating system 20 is self-contained and self-powered, without requiring a direct electrical connection to either the generator or the electrosurgical instrument. The self-powered arrangement shown in FIG. 6 can also be employed in the embodiment of FIG. 5, wherein the indicating system 20 is incorporated into the surgical instrument itself (e.g., within the housing of the electrosurgical instrument). In one embodiment the power supply 24 is a battery (e.g., a disposable coin battery, also known as a button cell). In other embodiments a rechargeable battery is employed wherein the battery is recharged by harvesting power from the RF current, using a photovoltaic cell, or by periodically supplying power to the indicating system from an external source (e.g., via an electrical outlet). In alternative embodiments, the indicating system is configured to receive power from an external source (e.g., FIG. 25, depicting an external power supply port) or even from the generator or the electrosurgical instrument. In the embodiment of FIG. 6, indicators 23 include an LED 27 and a buzzer 28. Of course, any of a variety of other tactile, visual and/or audible signaling devices or methods can be employed.

The controller circuitry 22 is configured to control activation of the indicator(s) 23 (e.g., LED 27, audible buzzer 28 or other devices such as a vibrator for tactile feedback) based on the RF current-indicative signal from the GMR sensor 21. The controller circuitry can be configured in a variety of ways, using a variety of components, in order to, for example, determine or estimate when the RF current is substantially constant and change the state of (e.g., activate) one or more of the indicator(s) 23. Generally speaking, the GMR sensor (e.g., in the form of an IC chip) is configured to provide a voltage signal that is proportional to the RF current through the tissue. In some instances (e.g., FIG. 6A) the GMR sensor is responsive only to the positive crest of the alternating RF current, thereby providing a voltage signal that, after filtering out the high frequency component, is proportional to the peak (or crest) value, and hence the magnitude of the RF current. In other instances (FIG. 6B) the GMR sensor is responsive to both the positive and negative crests of the alternating RF current, however, the negative crest is filtered out along with the high frequency component of the GMR signal (as further explained herein).

The voltage signal from the GMR sensor is then amplified and the time derivative of that signal determined. After filtering to remove high frequency noise, the voltage signal, representing the rate of change of the RF current through the tissue, is compared to a predetermined threshold. In on embodiment, if this differential voltage signal is at or below a threshold, or within a predetermined range of a set point, for at least a predetermined period of time, tissue treatment is deemed adequate and the state of one more indicators is changed (e.g., an LED is turned on). In the embodiment of FIG. 6A described below, a comparator is used to compare the differential voltage to a reference. In the embodiment of FIG. 6B described below, the comparison is performed by the microprocessor.

In the embodiment depicted schematically in FIG. 6, the controller circuitry 22 includes an amplifier & differentiator device 29 (also referred to as an “active differentiator” or “op-Amp differentiator”), a comparator device 25, and a processor in the form of a microprocessor 26. The microprocessor can be in the form of a microcontroller, or can have separate memory, analog-to-digital converter(s), and I/O devices not shown in FIGS. 6, 6A or 6B. Alternatively, an analog circuit or other device or circuitry capable of logically controlling actuation of the indicators can be used in place of (or in addition to) microprocessor 26.

As is known to those skilled in the art, a differentiator is a circuit that is designed to provide an output signal that is proportional to the rate of change (the time derivative) of its input, and an active differentiator is one that also includes an amplifier. The op-amp differentiator 29 outputs a differential voltage that is proportional to the rate of change of the voltage signal provided by the GMR sensor 21. The op-amp differentiator 29 also amplifies the differential voltage to a usable level (e.g., a gain of about 40 times). Therefore, the signal from the differentiator & amplifier device 29 is a differential voltage that is proportional to the change in the RF current relative to time (i.e., the first time derivative of the RF current that is flowing through the tissue via the RF current trace 31). The signals to and from the op-amp differentiator can also be filtered in order to remove the high frequency component of the voltage signal from the GMR sensor 21 as well as noise, as explained in connection with the description of the embodiment of FIG. 6B. In alternative embodiments, the amplifier can be provided as a device or circuit that is separate from the differentiator. Also, a signal conditioner or filter (e.g., at 38 in FIG. 6A) can be provided between the amplifier & differentiator device 29 and the comparator 22 in order to filter high frequency noise out of the differential voltage signal provided by the differentiator & amplifier device 29.

A variety of differentiator circuits are known to those skilled in the art. In one particular embodiment, indicating system 20 of FIG. 6A utilizes differentiator circuitry comprising a Wheatstone bridge circuit that causes a change in current through a capacitor. The capacitor's output voltage changes by creating current in the circuit; that is, the capacitor either charges or discharges in response to a change in applied voltage. The more capacitance the capacitor has, the greater its charge and discharge currents will be for any given rate of voltage change across it. Therefore, the voltage output from the differentiator circuit is proportional to the rate of change in the current applied to the tissue and this voltage output is continuous.

The op-amp differentiator 29 not only amplifies the GMR sensor output voltage, it also conditions (i.e., filters) the GMR sensor output voltage in order to filter out high frequency components of the GMR sensor output voltage, including high frequency noise. Such noise is picked up from the noise in the high frequency RF current and is passed through by the magnetic field to the GMR sensor or through coupling factors. In some embodiments, the controller circuitry is designed to provide a limited frequency response in order to improve the quality of the differential voltage signal (i.e., the output voltage from the differentiator circuit) by filtering out high frequency noise, as well as to provide a time delay. In particular, the controller circuitry in FIG. 6A is configured to provide a small time delay, with the length of the delay selected and designed in the circuitry to, for example, assure that the indicating system is not triggered due to a brief electrical short or an inflection point that might otherwise be mistaken as a stable RF current (e.g., when the current stops increasing and starts decreasing, such as the first zero crossing in FIG. 2). The time delay should be sufficient to avoid false detection but not add significantly to the total treatment time. By way of one example, the time delay can be at least about 100 milliseconds, from about 100 to about 1000 milliseconds, from about 150 to about 750 milliseconds, from about 200 to about 500 milliseconds, or about 200 milliseconds.

In the embodiment of FIG. 6A, the time delay as well as the filtering of high frequency noise is provided, for example, using a resister-capacitor (RC) circuit incorporated into the differentiator circuitry and/or provided downstream of the differentiator circuit. The RC circuit is a capacitor and a resistor combination such that the capacitor will discharge its stored energy through the resistor when the output of the differentiator reduces to a level less than the charge on the capacitor. The voltage across the capacitor is time dependent and will delay the reduction of the output. The incorporation of the time delay, for example, simply assures that the voltage signal from the GMR sensor must remain stable, or alternatively the rate of change of the voltage signal must be below a predetermined threshold, for a predetermined period of time (e.g., 200 milliseconds) before the indicating system triggers the indicator(s).

In the embodiment of FIGS. 6 and 6A, the differential voltage, which is representative of the rate of change of the RF current through the tissue, is supplied by the op-amp differentiator to a comparator 25, which determines if the differential voltage is high or low in comparison to a selected threshold or trip voltage. A low differential voltage indicates that the RF current has been stable, or alternatively that the rate of change of the voltage signal from the GMR sensor, has been below a predetermined threshold, for at least the predetermined time delay). The comparator 25 compares the conditioned differential voltage from the op-amp differentiator to a predetermined threshold voltage in order to determine if the RF current is substantially stable. The predetermined threshold voltage provides a trip point for activating the indicators 23 when the conditioned differential voltage provided to the comparator is below the threshold voltage trip point. The predetermined threshold voltage is based on the comparator device manufacturer's recommendations or may be empirically derived to assure adequate sensitivity (e.g., at or near zero indicating that the RF current is stable). In one particular embodiment, the differential voltage trip point is 400 mV (millivolts) based on design of the circuits or preset by the manufacturer of the comparator device. However, the controller circuitry can be designed to use any voltage trip point.

When the comparator 25 receives a differential voltage below the threshold trip point it signals the microprocessor 26 to actuate an indicator (e.g., indicator 28) in order to signal the user that the treatment (e.g., vessel sealing) is complete. In some embodiments this occurs when the RF current is at or near steady state, indicating the tissue is adequately sealed or cauterized. In other embodiments this occurs a predetermined period of time after the rate of change in the voltage signal falls below a predetermined level (e.g., at the end of Stage 1 in FIG. 1). In still further embodiments, multiple trip points may be included to detect, for example, an electrical short and/or to control switching off the flow of RF current through the tissue. For example, in some embodiments, a trip point can be used to signal the start of a timer to record the total elapsed time that current is flowing through the tissue, and then signal the user when a predetermined period of time has elapsed. It will be understood that an analog circuit, an application specific integrated circuit(s) (ASIC), and/or a field programmable gate array can be used in place of the microprocessor 26.

In one particular embodiment, the indicating system includes two indicators (e.g., an LED 27 and a buzzer 28). The first indicator (e.g., the LED 27) is activated when RF energy is applied to the tissue and a current is flowing above a nominal set threshold, causing the differential voltage supplied to the comparator to be above the trip point of the comparator and triggering the microprocessor to activate the first indicator (e.g., Stage 1 of tissue treatment). As the RF current through the tissue is reduced, such as to a steady state, the output of the differentiator falls to below the predetermined threshold voltage of the comparator. With a predetermined delay due to the decaying of the stored energy on the capacitor of the RC circuit, the differential voltage will decrease to below the trip point of the comparator, causing the first indicator to be deactivated and causing the second indicator to be activated. The second indicator may be, for example, audible (e.g., buzzer 28) to signal the user that the treatment is completed.

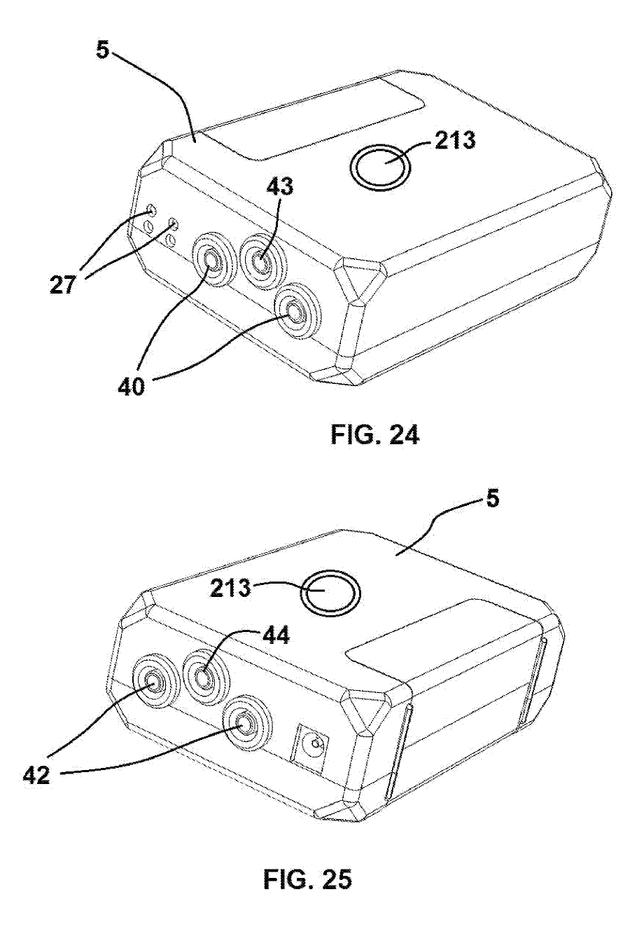

FIG. 6A provides a more detailed block diagram of one embodiment of indicating system 20 of FIG. 6, configured for being located between an RF generator and an electrosurgical instrument (e.g., electrosurgical forceps). Thus, as seen in FIGS. 24 and 25, indicating system 20 is provided in a housing 5 having a first set of connectors 40 for operatively connecting a cable between the indicating system 20 and the electrosurgical forceps (or other instrument), and a second set of connectors 42 for operatively connecting a cable between the indicating system 20 and an RF generator 30. A button (or other form of switch) 213 is also provided on the housing for activating the indicating system. Connectors 40 and 42 are used for communicating the RF current between the generator and the forceps. Connectors 43 and 44 are provided on the housing for passing a hand switch sense signal from the instrument to the generator in order to, for example, detect that a handpiece (i.e., the instrument) is connected. This also allows the hand switch on the instrument to be used to activate the delivery of electrosurgical energy from the generator to the instrument. In the example shown, the hand switch sense signal is simply passed through the housing, via an electrical conductor that provides electrical communication between connector 43 (to the instrument) and connector 44 (to the generator).

Indicating system 20 in FIG. 6A further includes a switch 37, actuable by button 213 on the housing 5, for activating (turning on) the indicating system 20. In some embodiments, and as described in connection with FIG. 6B, switch 37 can be a switch logic device (also referred to as a logic switch) controllable by the microprocessor 26 such that power to the indicating system is shut down after a predetermined period of time following initial startup and/or use (e.g., two hours) in order to save battery life. Indicating system 20 further includes signal-conditioning circuitry 38 (which includes an RC circuit time delay) between the op-amp differentiator 29 and the microprocessor 26. The 400 mV voltage reference 34 of comparator 25 is also depicted in FIG. 6A, along with an LED timer set 35 for establishing the maximum time of LED activation (to save battery power).

FIG. 6B provides a block diagram of an alternative embodiment of indicating system 20, wherein the system is configured to be incorporated into an electrosurgical instrument (as further described herein). In the embodiment of FIG. 6B, the microprocessor 26 is responsible for analyzing the filtered and differentiated GMR sensor signal, as provided by the op-amp differentiator 29 in response to the signal from the GMR sensor 21, to determine if the RF current is stable as well as controlling the activation of the indicators 27 and 28. Thus, the microprocessor 26 in FIG. 6B performs the functions of, and is therefore used in place of, the LED Timer Set 35 and comparator 25 of the embodiment of FIG. 6A.

Microprocessor 26 in FIG. 6B is adapted (i.e., programmed) to not only determine when the RF current through tissue is stable and activate one more indicators upon such a determination, but also to monitor the progress of tissue treatment (e.g., vessel sealing or tissue cauterization), initialize the system at startup, maintain power to the indicating system for a predetermined period of time following startup, cancel out system noise and ensure accuracy of the sensor readings.

While the GMR sensor 21 in FIG. 6B can be configured like that used in FIG. 6A to be responsive only to the positive crest of the AC RF current, GMR sensor 21 in FIG. 6B is responsive to both the positive and negative crests of the AC RF current (i.e., the GMR sensor in FIG. 6B is bipolar). GMR sensor 21 in FIG. 6B thus provides two output signals—one representing the positive crests of the RF current and the other representing the negative crests of the RF current. Supply voltage (Vcc) is applied across a resistor to the GMR sensor 21 in order to add a DC positive bias to the GMR sensor output. As before, the voltage signal provided by the GMR sensor 21 has a high frequency component due to the AC nature of the RF current, and a low frequency component from change in magnitude of RF current as tissue treatment progresses. Only the low frequency component is of interest for purposes of monitoring the progression of tissue treatment.

In order to filter out the high frequency component and other noise in the voltage signal from the GMR sensor in FIG. 6B, the negative output of the GMR sensor 21 is supplied to the inverting input of the op-amp differentiator 29 through a filter capacitor, while the positive output of the GMR sensor 21 is supplied to the non-inverting input of the op-amp differentiator 29 through a smaller filter capacitor. The filter capacitors filter out high frequency components of the GMR sensor output, and the use of a larger filter capacitor for the negative output of the GMR sensor effectively filters out the negative crest of the AC voltage signal of the GMR sensor. This provides for the mitigation of some of the noise that is inherently coupled into the electronic circuits when RF current is applied to the tissue, and also ensures that the output of the op-amp differentiator 29 will be a positive output above what could be a noisy environment. An RC circuit in the op-amp differentiator 29 also provides additional filtering of high frequency components, including noise, such that the op-amp differentiator 29 amplifies the low frequency component of the differential voltage significantly more than any remaining high frequency components. Thus, the op-amp differentiator 29 provides a slow changing (i.e., low frequency), positive voltage signal representing the rate of change in the RF current through the tissue over time. As in FIG. 6A, a signal conditioning circuitry 38 located between the op-amp differentiator 29 and the microprocessor 26 further filters the output of the op-amp differentiator 29, and provides an analog signal to the ADC input 26A of the microprocessor. This analog signal is effectively the signal represented by FIG. 2, with a positive bias such that the signal remains positive. This signal is also proportional to the rate of change of the RF current through the tissue and is used by the microprocessor to monitor tissue treatment (as further described below).

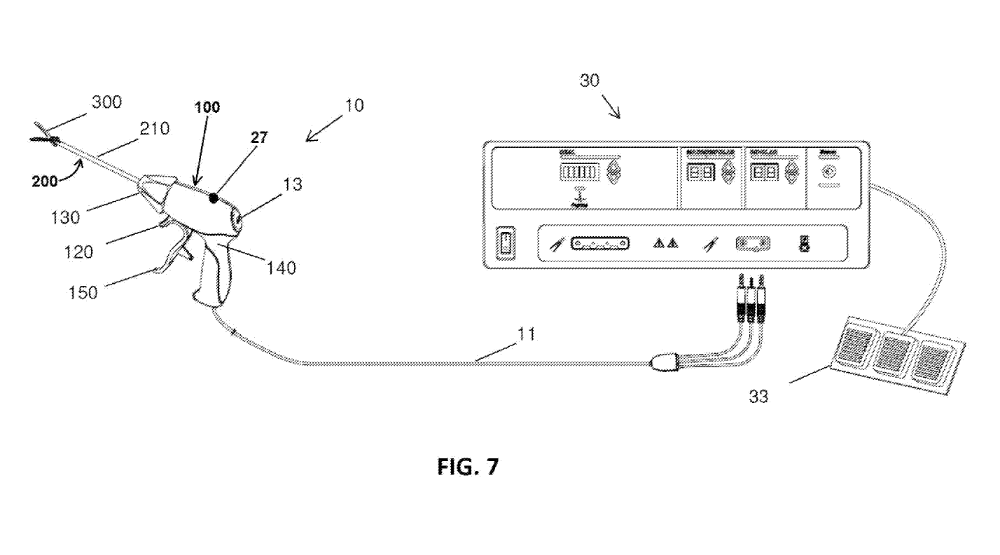

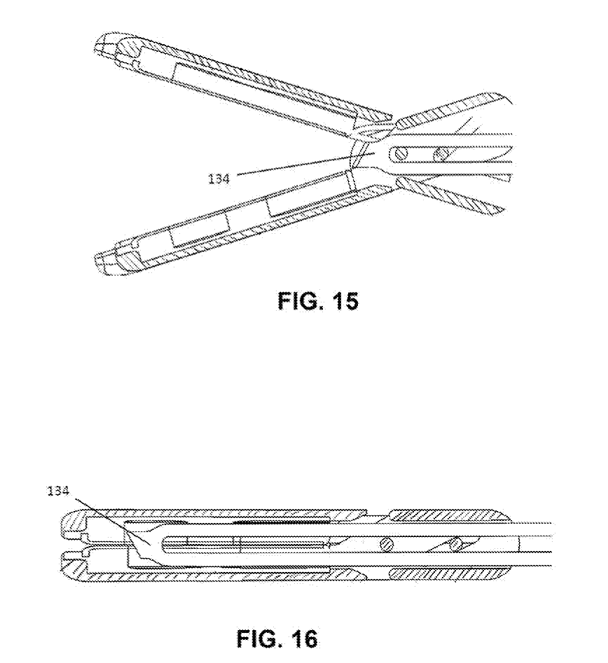

Further noise mitigation is provided by the microprocessor 26 in the embodiment of FIG. 6B. Noise may be inherently coupled in the electronic circuits of the indicating system 20 and related equipment such as the generator 30. Such noise, if not mitigated, could cause the microprocessor 26 to falsely trigger the LED 27 and buzzer 28, particularly if the noise exceeds the signal level resulting from the GMR sensor 21 (as processed by the op-amp differentiator 29 and signal conditioner 38). In addition, to the noise mitigation in the processing of the signal from the GMR sensor 21 described above, the microprocessor 26 in FIG. 6B also mitigates noise inherent in the indicating system 20, beginning when the indicating system 20 is first activated (i.e., powered, such as by activating switch 36). When the indicating system is first activated (e.g., by pressing activation switch 13 in the embodiment of FIGS. 7-23), and following a brief, predetermined time delay, a “snapshot” reading of the base line or noise level voltage on the ADC input 26A is recorded by the microprocessor 26. The predetermined time delay before the snapshot voltage is taken allows for stabilization of all the circuits within the device. This snapshot voltage level is defined by the tolerances and offsets of all of the components in the signal chain up to the analog to digital converter (ADC) input to the microprocessor 26. The snapshot voltage is stored in memory and is later used as a reference for all other processes in the operation of the device.

In the particular embodiment of indicating system 20 in FIG. 6B, the system includes both a generator enabling switch 36 and a logic switch 37. While these can be separate switches, in the depicted embodiment switches 36 and 37 are provided by a double detent tactile switch that is activated, for example, by pressing button 13 is provided on the handle of the instrument (see FIG. 13). When the user first activates button 13 (or other switch on the instrument or on the housing of a standalone indicating system), with the indicating system/instrument not connected to the generator, generator enabling switch 36 and logic switch 37 are closed (i.e., actuated), thereby providing battery power to Vcc and the microprocessor. The user only needs to press button 13 briefly to activate the indicating system. Thereafter, the microprocessor will maintain logic switch 37 closed (i.e., actuated) for a predetermined period of time such that battery power is continually supplied to the indicating system 20. In one embodiment, the microprocessor maintains logic switch 37 closed for at least one hour, or in some instances two hours, following startup or the last used of the instrument in treating tissue. Thus, the microprocessor maintains the indicating system in a ready state during a surgical procedure, without the need to restart and reinitiate the system each time that the surgeon desires to use the electrosurgical instrument. Shutting down after a period of non-use helps to preserver battery life. If the indicating system 20 powers down during a surgical procedure due to an extended period of non-use, the indicating system can be reinitiated by pressing button 13. As further discussed below, when button 13 is pressed and held down, with a generator operatively connected to the instrument, electrosurgical energy will be delivered from the generator to the instrument. In this instance, generator enabling switch 36 will send a hand switch signal to the generator, as further described below. It will be understood that the generator enabling switch 36 and the logic switch 37 may be separate or combined in a double pole, single or double throw, switch that is actuated by a button (e.g., button 13) or other actuator located on the instrument or, in the case of a standalone system, on the indicating system housing. Alternately, the switch action could be performed by separately actuated switches if desired.

Following initial supply of power to the microprocessor, the indicating system 20 of FIG. 6B will undergo an initialization process. In one embodiment, the LED 27 flashes rapidly and the buzzer 28 emits a rapid series of beeps indicating that system startup has commenced. If desired, the LED 27 also flashes so as to indicate the version number of the control circuitry software. Of course these indicator actuations at system initialization are merely exemplary of one possible configuration. After the brief, predetermined time delay (e.g., about 0.25 to about 0.5 seconds), the snapshot reading of the voltage on the ADC input 26A of the microprocessor 26 is recorded by the microprocessor 26. This occurs prior to the generator supplying RF current to the instrument, and an additional visual and/or audible indication can also be provided to the user indicating that the instrument is ready to be used in treating tissue.

Following startup and system initialization, the instrument is ready to be used in treating tissue. After the generator is connected to the instrument via a cable 11, the surgeon can press and hold button 13, thereby providing a hand switch signal to the generator to initiate the supply of RF current to the instrument as long as the button 13 is pressed. In the alternative embodiment of a standalone indicating system, the system can be configured to pass through a hand switch signal from an attached instrument to an attached generator. Of course a foot switch or other actuating device can also be used to initiate the flow of RF current to the end effector of the instrument.

When the generator enabling switch 36 is held closed (e.g., by pressing and holding button 13), a hand switch signal is sent to the generator. In response, the generator delivers electrosurgical energy to the instrument (at a constant voltage). In some instances, the hand switch signal is simply the voltage of the power source in the indicating system 20, which is delivered to a hand switch sense port on the generator. In the embodiment shown in FIG. 6B, an optocoupler 36B is used to cause a low voltage AC signal, taken from the high current trace through a resistor (e.g., 4000Ω resistor, not shown), to be passed back to the generator via a connector 44 to which the generator is operatively connected via cable 11 in FIG. 6B as long as the generator enabling switch 36 is held closed. An optocoupler 36B is used for this purpose in order to isolate the indicating system 20 from the RF current.

Following startup and initialization of the indicating system 20 of FIG. 6B, when button 13 is pressed and held down (or a foot switch connected to the generator is activated), RF current will flow through the high current trace 31. The GMR sensor 21 provides the voltage signal that is proportional to the current flowing through the trace 31 to the tissue. As discussed above, the voltage signal from the GMR sensor 21 is filtered to remove high frequency components and noise, and then differentiated to provide a voltage signal proportional to the rate of change of the RF current flowing through the tissue. This signal is supplied to the ADC input 26A of the microprocessor, and is converted into a digital signal for processing and to control visual and audible indicators such as LED 27, buzzer 28 or other devices such as a vibrator for tactile feedback. Indicator devices such as the buzzer 28 may require a boost power supply 28B to ensure adequate audible output.

The microprocessor 26 processes amplified, conditioned and digitized voltage signal provided by the GMR sensor 21 to monitor tissue treatment. While in some embodiments this can comprise simply monitoring for a steady date voltage, the embodiment of FIG. 6B is configured such that the microprocessor 26:
a) determines that sealing or other tissue treatment has commenced based on whether the voltage signal is equal to or exceeds a first predetermined threshold, thereby indicating that the tissue treatment is in Stage 1 (FIG. 1);
b) determines that tissue treatment is in Stage 2 based on, for example, the voltage signal being equal to or less than a second predetermined threshold (e.g., to identify when the RF current is decreasing);
c) determines that tissue treatment is adequate (i.e., Stage 3 has been reached) based on the voltage signal being within a predetermined range around a third predetermined threshold above the snapshot voltage, for a predetermined period of time—thereby indicating that the RF current is stable; and
d) causes one or more indicators to change state (e.g., activates or deactivates LED 27 and/or buzzer 28) thereby indicating to the user that an adequate seal or cauterization has been achieved based on a determination that the RF current has stabilized (e.g., by flashing the LED and sounding the buzzer or other means such as vibration).
   For each of the above determinations (a)-(c), the microprocessor 26 of the embodiment in FIG. 6B is configured to use the snapshot voltage acquired during system initialization as a baseline reference voltage in order to factor out noise inherent in the indicating system (e.g., by subtracting the snapshot voltage from the voltage signal).

In some instances, the microprocessor can be further configured to alert the user if one or more errors or other irregular conditions are determined. For example, the microprocessor can be adapted to identify when the application of RF current to tissue has commenced (e.g., as the time when a signal above the snapshot voltage is received on ADC input 26A). This can then be used to monitor how long the treatment takes to one or more of the three treatment stages, and providing an error indication to the user if one of those time periods meets or exceeds a predetermined duration of time. The microprocessor also can be adapted to identify if a voltage signal received by the ADC input 26A is outside of predetermined level or range during one or more of the stages. For example, if the voltage signal in Stage 1 is too high (i.e., the current through the tissue is rising abnormally rapidly), an error signal can be provided as an indication of a short. Similarly, if the voltage signal in Stage 2 is too low (i.e., the current through the tissue is falling abnormally rapidly) an error signal can be provided as an indication of, for example, tissue not being present between the jaw members of electrosurgical bipolar forceps.

For purposes of determining when the RF current is stable or when the treatment has reached Stage 1 or Stage 2, the microprocessor is configured to average the digitized voltage signals over a small period of time in order to avoid spurious activation of an indicator(s) due to, for example, slight variations in the voltage signal that is indicative of the rate of the change in RF current—particularly since the rate of change of the RF current will never be precisely zero over any period of time. For example, the microprocessor can be configured to average the digitized voltage signal over a small interval (e.g., 5-10 samples over a period of 150 to 350 milliseconds, or 8 samples over 250 milliseconds) to provide more appropriate data points for determining the various treatment stages and/or when an error condition exists.

With respect to the determination of when Stage 3 is reached (i.e., RF current is stable and therefore treatment is adequate), it can be important to verify that the rate of change of the GMR voltage (and hence RF current) is substantially zero (i.e., within a predetermined range±zero) over a predetermined period of time. In the embodiment of FIG. 6B, this is accomplished by the microprocessor monitoring the digitized voltage signal, averaged in the manner noted above (e.g., 5-10 samples over a period of 150 to 350 milliseconds, or 8 samples over 250 milliseconds), until that voltage remains within a predetermined range (e.g., ±200 mV, ±100 mV, or ±50 mV) about a predetermined set point for a predetermined period of time. For example, the microprocessor averages 5-10 samples of the digitized voltage signal over a period of 150 to 350 milliseconds (e.g., 8 samples over 250 milliseconds), and then determines if each of those averaged values during a predetermined period of time is within a predetermined range of the constant current set point. In this instance, the predetermined set point represents the digitized voltage signal that represents a zero rate of change in RF current (i.e., a truly constant RF current). The set point itself is not a zero voltage, however, due to the manner in which the controller circuitry processes the GMR sensor signal (i.e., the voltage signal provided by the op-amp differentiator is always greater than zero). In the embodiment of FIG. 6B, the predetermined time period during which the voltage signal must be within a predetermined range before the tissue treatment is deemed adequate is at least about 200 milliseconds, from about 200 to about 2000 milliseconds, from about 500 to about 1500 milliseconds, from about 1000 to about 1400 milliseconds, or about 1250 milliseconds. For example, when the microprocessor averages 8 samples of the digitized voltage over 250 milliseconds, the microprocessor deems tissue treatment to be adequate when each of a sequence of five such averaged values (representing a total of 1250 milliseconds of treatment time) are all within the specified range of the set point (e.g., ±100 mV).

The embodiment depicted in FIG. 6B of the current disclosure utilizes software, via the microprocessor, to provide greater flexibility, capability, accuracy and reliability for timing, monitoring, and processing data for controlling the indicating system. In some embodiments, this indicating system also can be utilized to control the ESU 30 by providing one or more signals thereto.

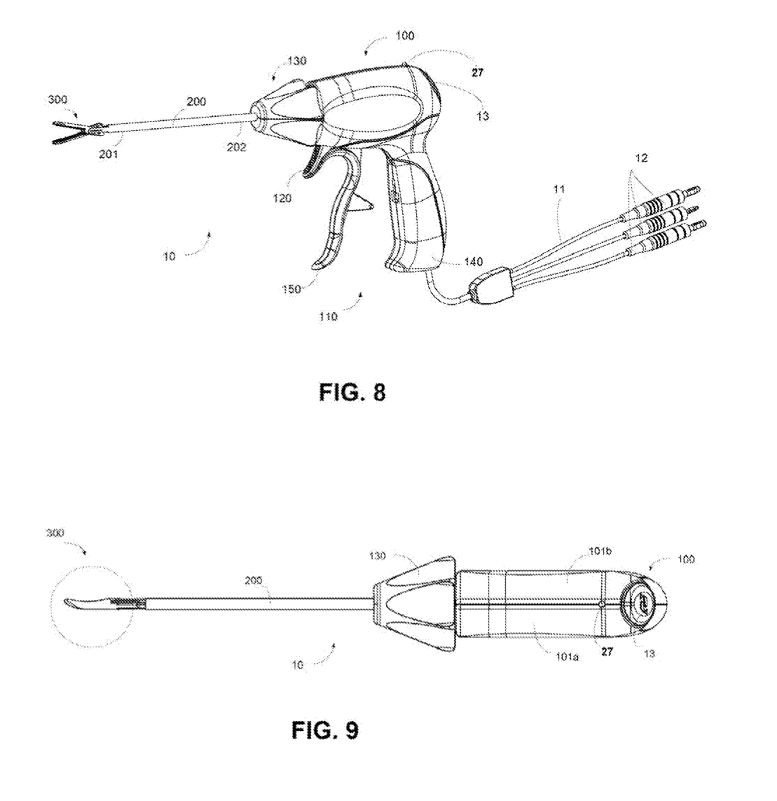

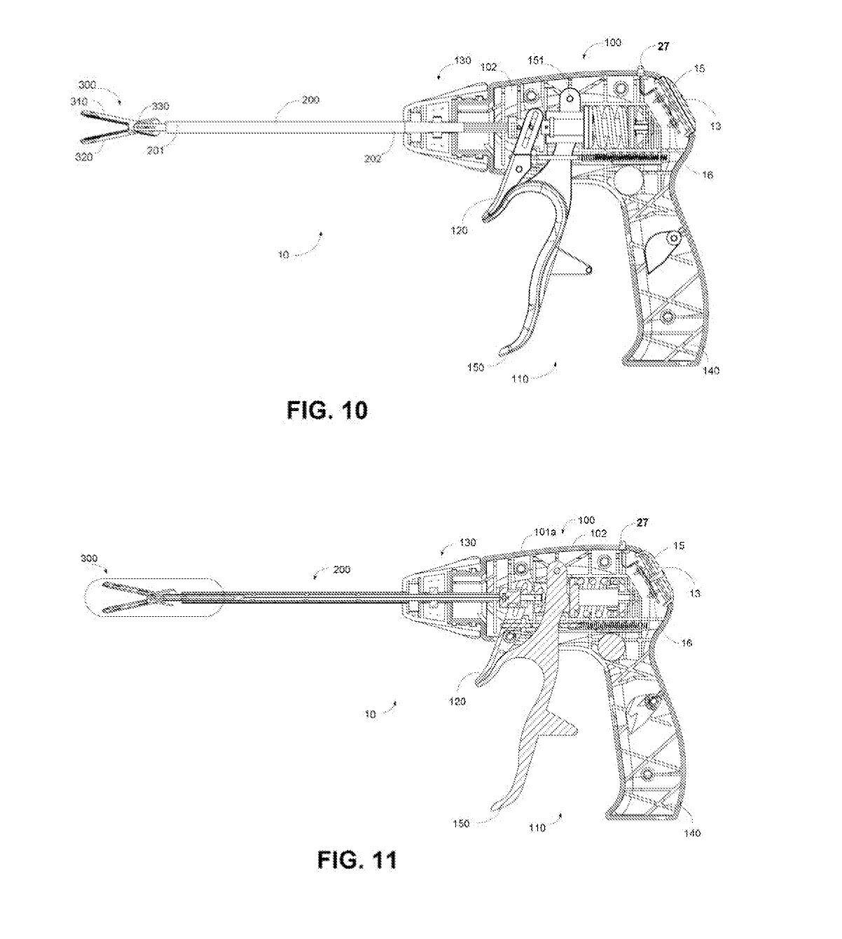

As mentioned previously, the embodiment of indicating system 20 depicted in FIG. 6B is configured to be incorporated into the electrosurgical instrument itself (as further explained below). By way of example, and as further described below, in an alternative embodiment the indicating system 20 (e.g., FIG. 6A or FIG. 6B) is mounted on a circuit board 16 (see FIGS. 10-12) and housed within a handheld electrosurgical instrument 10. Alternatively, this same system can be incorporated within the generator 30, cable 11 (see FIG. 7), a connector 12 (see FIG. 8).

As yet another alternative, the indicating system of FIG. 6B can be configured as a standalone unit for being operatively connected between a generator and the surgical instrument, such as shown in FIGS. 4, 24 and 25. In such an alternative embodiment, it would be allow the use of a hand switch on the instrument for controlling the delivery of electrosurgical energy to the instrument. desirable to provide the Since it may be imp

FIGS. 7-23 depict an electrosurgical system similar to that of FIG. 5, wherein the indicating system 20 of FIG. 6B is incorporated into the housing of electrosurgical forceps 10. FIG. 7 depicts the forceps 10 along with an RF generator 30 to which the forceps can be operatively attached via a cable 11. As depicted in FIG. 7, the RF generator 30 includes a foot switch 33 for controlling operation of the generator.

The bipolar forceps 10 of FIGS. 7-23 generally includes a cable 11 (shortened in FIG. 8 for purposes of clarity), a housing 100, an activation button 13 located, for example, on a rear face of the housing, a visual indicator 27 (e.g., an LED), an elongated portion 200 and an end effector 300. While cable 11 is depicted as being integral with (i.e., permanently affixed to) the instrument, it will be understood that the instrument and cable can be configured such that the cable is detachable from the instrument using connectors 42 (2x) and 44 in FIG. 6B. The distal end 201 of the elongated portion 200 is in connection with the end effector 300, and the proximal end 202 of the elongated portion 200 is in connection with the housing 100 of the bipolar forceps 10. The housing 100 includes a handle assembly 110 configured to be grasped by a surgeon, a movable handle 150, a knife trigger 120 and a rotating assembly 130. It will be understood that the electrosurgical instrument shown in FIGS. 7-12 is exemplary of one embodiment, and the indicating system of the present disclosure can be incorporated into any of a variety of other electrosurgical instruments having a variety of different configurations.

The handle assembly 110 includes a stationary (e.g., fixed) handle 140 and at least one movable handle 150. The stationary handle 140 is integrally associated with (e.g., molded with) the housing 100. As best seen in FIGS. 9 and 10, the housing 100 includes two halves 101a and 101b that, when assembled, form an internal cavity 102. The movable handle 150 is pivotally mounted within the internal cavity 102 of the housing 100, with the movable handle 150 pivotable about a pivot pin 151.

The end-effector 300 is provided with a pair of jaw members 310 and 320, selectively positionable relative to one another about a pivot 330. The end effector 300 is configured for grasping, dissecting and/or clamping tissue, such as for constricting vessels for purposes of sealing. Each of the jaw members 310 and 320 has an electrically conductive tissue-engaging surface such that RF current can be conducted from one jaw member to the other through tissue clamped between the jaw members.

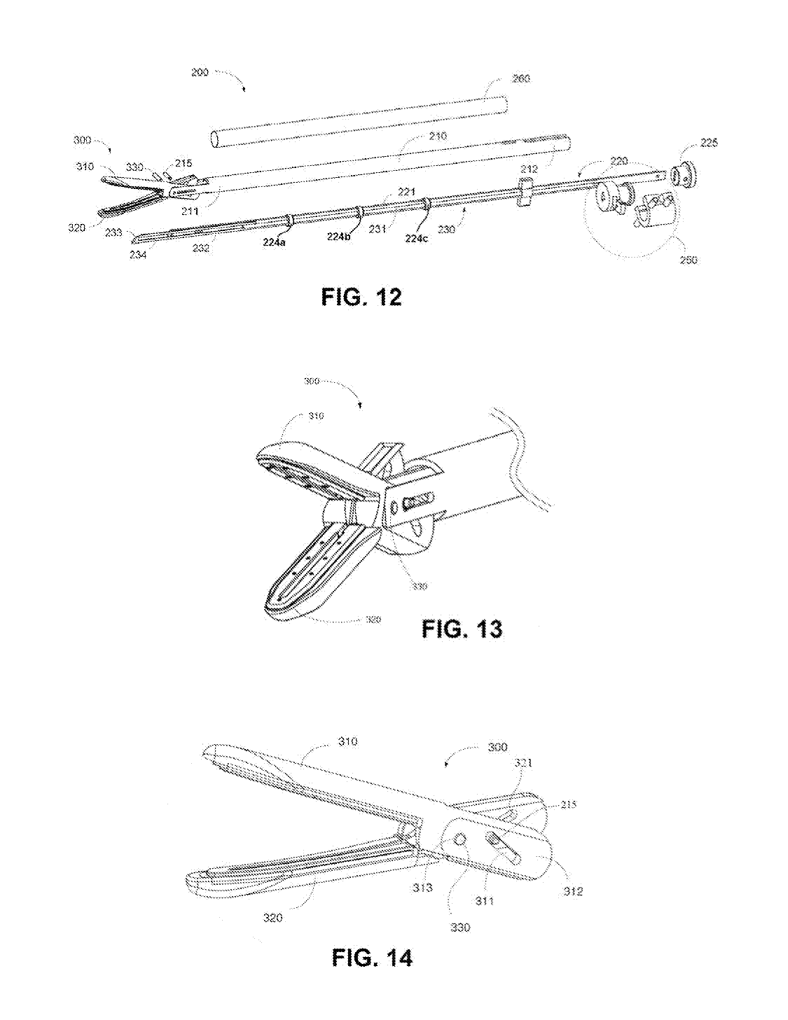

The movable handle 150 of the handle assembly 110 is operatively connected to a drive assembly 220 (see FIG. 12) of the elongated portion 200. The movable handle 150 and drive assembly 220 together mechanically cooperate to impart movement of the jaw members 310 and 320 from an open position wherein the jaw members 310 and 320 are disposed in spaced relation relative to one another, to a clamping or closed position wherein the jaw members 310 and 320 cooperate to grasp tissue therebetween.

The end-effector 300 is coupled to the distal end of the elongated portion 200. In the depicted embodiment, the pair of jaw members 310 and 320 is configured for grasping, dissecting and/or clamping tissue and further includes at least one delivery system for delivering RF energy to the tissue, wherein the RF energy is supplied by an RF generator operatively connected to the forceps 10. Each of the jaw members 310 and 320 therefore includes electrodes 316 (see FIG. 19) that comprise electrically conductive tissue engaging surfaces adapted to be operatively connected to the RF energy source (i.e., such that current passes from one electrode to the other, through tissue clamped between the jaw members).

As best illustrated in FIGS. 12 and 19, the elongated portion 200 of the bipolar forceps 10 includes an insulating tube 260 and a shaft 210 having a distal end 211 dimensioned to mechanically engage the end effector 300. Insulating tube 260 is assembled over shaft 210, and provides additional insulation in case of an electrical short. Referring to FIG. 19, the shaft 210 is bifurcated at the distal end 211 thereof to form ends 212a and 212b that are dimensioned to receive the end effector 300. Accordingly, the opposing jaw members 310 and 320 of the end effector 300 are seated between the bifurcated ends 212a and 212b of the shaft 210. The shaft 210 also includes a pair of longitudinally oriented slots 214a and 214b disposed on the bifurcated ends 212a and 212b, respectively. The slots 214a, 214b are dimensioned to allow the longitudinal reciprocation of a pin 215 located therein. This longitudinal reciprocation of pin 215 causes movement of the opposing jaw member 310 and 320 between their open and closed positions. The shaft 210 also includes a pair of holes disposed in the bifurcated ends 212a and 212b, which are dimensioned to receive a pivot pin 330 that is adapted for securing the jaw members 310 and 320 to the shaft 210 between the bifurcated ends 212a and 212b while still allowing the jaw members 310 and 320 to pivot. Longitudinal reciprocation of the pin 215 within the slots 214a, 214b causes the jaw members 310 and 320 to rotate about the pivot pin 330 from the open to closed positions and to reopen the jaw members 310 and 320.

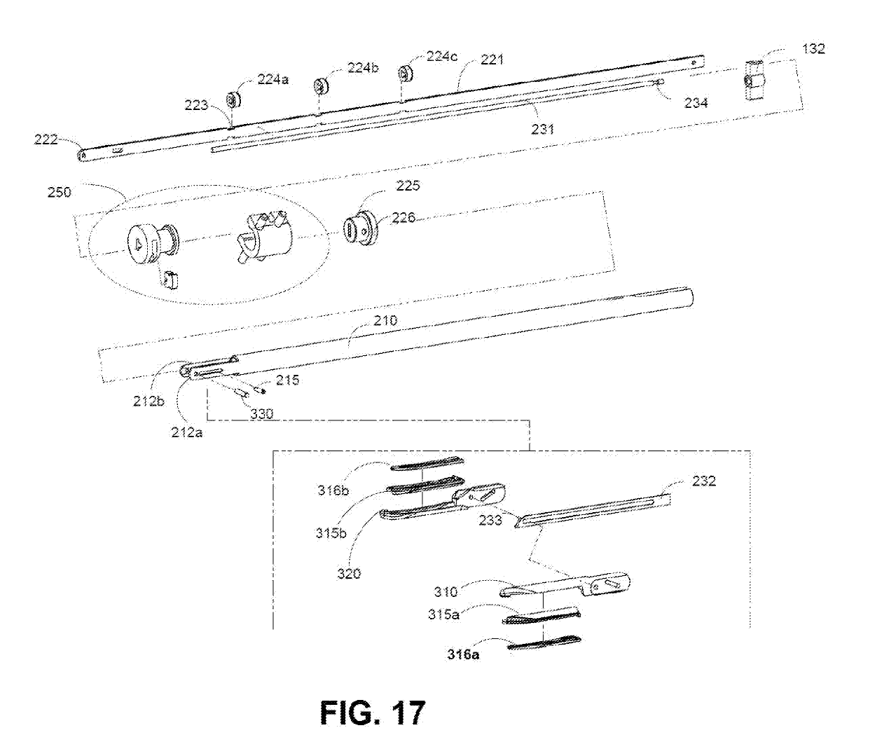

Accordingly, referring to FIGS. 13 and 14 (wherein FIG. 13 depicts a straight end effector alternative), the jaw member 310 of the end effector 300 includes a cam slot 311 arranged on the proximal end 312 thereof, wherein the cam slot 311 is dimensioned to engage the pin 215 such that longitudinal movement of the jaw driver 221 (FIG. 17) of the jaw closing assembly 220 causes the pin 215 to ride along the cam slot 311. The distal end of the jaw driver 221 includes a hole 222 (see FIG. 17) for receiving the pin 215. The jaw member 310 also includes a hole 313 arranged on the proximal end 312 thereof, which is dimensioned to receive the pivot pin 330 (see FIG. 14). Likewise, the jaw member 320 also includes a cam slot 321, a hole for receiving the pin 313, such that longitudinal movement of the jaw driver 221 of the jaw closing assembly 220 causes the pin 215 to ride along both the cam slots 311 and 321, resulting in the opposing jaw members 310 and 320 rotating about the pivot pin 330 between their open and closed positions. A variety of end effector arrangements can be provided, including the curved end effector depicted in FIGS. 9, 14 and 19, and the straight end effector depicted in FIG. 13.

Referring to FIGS. 11 and 12, and as mentioned previously, the elongated portion 200 of the bipolar forceps 10 includes a jaw closure assembly 220 configured to cooperate with the movable handle 150 to impart movement of the jaw members 310 and 320 between the open and closed positions. The elongated portion further includes a knife assembly 230 configured to cooperate with the knife trigger 120 to incise tissue clamped between the jaw members 310 and 320 after vessel sealing or tissue cauterization.

More specifically, as shown in FIGS. 11, 12, 20 and 21, the housing 100 encloses the proximal portion of the jaw closing assembly 220 which cooperates with the movable handle 150 to impart movement of the jaw members 310 and 320 between an open position wherein the jaw members 310 and 320 are disposed in spaced relation relative to one another, and a clamping or closed position wherein the jaw members 310 and 320 cooperate to grasp tissue therebetween. The housing 100 also encloses the proximal portion of the knife assembly 230, which cooperates with the knife trigger 120 to reciprocate the knife 232 disposed on the distal end of the knife assembly for incising the tissue clamped between the jaw members 310 and 320. The distal end of the knife assembly 230 is disposed between the opposing jaw members 310 and 320 of the end effector 300.

The knife assembly 230 and the end effector 300 are independently operable. The knife trigger 120 actuates (i.e., moves distally) the knife assembly 230 while the movable handle 150 actuates the closing and opening of the jaw members. More specifically, referring to FIGS. 12 and 17, the knife assembly 230 includes a knife rod 231 and a knife 232 having a cutting edge 233 on the distal end thereof. While the knife rod 231 and knife 232 can be integrally formed from, for example, a single piece of metal, in the depicted embodiment the proximal end of the knife 232 is welded to the distal end of the knife rod 231. The knife assembly 230 is arranged on one side of the jaw driver 221 of the jaw closing assembly 220, with a plurality of sealing rings 224a-224c retaining the knife rod 231 along one side of jaw driver 221 such that the knife rod 231 can be selectively reciprocated independently of and with respect to the jaw driver 221. As best seen in FIG. 17, the jaw driver 221 comprises an elongate, plate-like member having a plurality of notches 223 arranged in its upper and lower edges. Each sealing ring 224a-224c is positioned within an aligned pair of the notches 223 for retaining the knife rod 231 alongside the jaw driver 221. The sealing rings 224a-c are also dimensioned to seal against the interior wall of the shaft 210 in order to maintain pneumo-peritoneal pressure during endoscopic procedures as well as prevent the inundation of surgical fluids which could be detrimental to the internal operating components of the forceps 10. The sealing rings 224a-224c are also dimensioned so as to allow the plate-like jaw driver 221, the knife rod 231 and the two RF current carrying wires 31 to pass therethrough so as to connect them therewith, while also allowing the jaw driver 221 and the knife rod 231 to reciprocate independently of each other.

Referring to FIGS. 17 and 19, each jaw member 310, 320 is assembled with a nonconductive insulator 315a, 315b, and a conductive electrode 316a, 316b that is electrically connected by an RF current carrying trace wire 31 that is in electrical communication with cable 11 such that the electrodes can be places in electrical communication with an RF generator to which cable 11 is connected. The pair of RF current trace wires 31 pass through the elongated portion 200 and the housing 100 to a circuit board 16, which is in electrical communication with the cable 11 that extends through and out of the stationary handle 140 for connection to an energy source 30. Likewise, the cable 11 caries one or more conductors from a hand switch 15 (see FIGS. 10 and 11) such that the hand switch 15 can be used to active and deactivate the energy source 30 and control the RF current. The cable 11 as well as the wire traces and other electrical connections are omitted from the internal views of the housing for the purpose of clarity.

The electrosurgical instrument 10 further comprises the indicating system 20 of FIG. 6B, disposed within the housing 100, which is adapted for providing a user with a signal indicating that coagulation or sealing is adequately completed. In addition, or as an alternative to signaling the user when the tissue is adequately sealed, the indicating system 20 can be configured to provide the user with a signal of other events such as when the total elapsed time that RF energy is applied through the tissue reaches a predetermined period of time, as described previously herein. The indicating system 20 is provided on a circuit board 16, which also includes a double detent tactile hand switch 15 corresponding to the switches 36 and 37 of FIG. 6B. A button 13 is used to actuate the hand switch 15.

The electrosurgical instrument 10 further includes an electrosurgical cable 11 (FIG. 8), which is used to connect the instrument 10 to an energy source 30 (e.g. an ESU) as a source of RF current or electrosurgical energy. The electrosurgical cable 11 extends to one or more connectors 12 including prong members, which are dimensioned to mechanically and electrically connect the electrosurgical instrument 10 to an energy source 30 such as an electrosurgical bipolar generator. Electrosurgical cable 11 may carry multiple conductors for the RF current and to control the energy source 30. For example conductors may be in electrical communication with hand activation switch 15 (FIG. 10) which is mounted on the circuit board 16 and positioned under activation button 13. When the button 13 is pressed, it activates switch 15, sending a signal to the energy source 30 via conductors in the cable 11 for activating the energy source 30 to supply electrosurgical energy to the instrument, as described previously with respect to FIG. 6B. Alternatively, the generator may be activated by a foot switch 33 or other means connected to or internal to the energy source 30. RF current is then connected via the cable 11 (FIG. 8) to the end effector electrodes 316 via RF current traces 31 (see FIG. 19). RF current trace 31 is an insulated wire and a separate RF current trace 31 or wire is connected to the opposite electrode 316 (see FIG. 17) such that RF current can be applied to tissue clamped between the jaw members 310 and 320 of the end effector 300. At least one RF current trace 31 leads past the sensor 21 of the indicating system 20 (e.g., FIGS. 5 and 6), on circuit board 16.

As discussed previously, the Applicants have discovered that tissue or blood vessels are adequately sealed, blood is coagulated, or other tissue is cauterized when the RF current is constant with respect to time (see FIG. 1). The controller of the indicating system is adapted to control the state of one or more indicators (e.g., LED 27) so as to indicate to the user that the electrosurgical treatment is adequate. The user may then discontinue the application of RF current to the tissue such as by pressing button 13 or footswitch 33. Alternatively, the indicating system can be configured to control the energy source 30 upon determining that tissue treatment is adequate. In a particular embodiment, the controller includes a microprocessor 26 configured to signal the user when the RF current at or near steady state (i.e., constant or stable) for a predetermined period of time (e.g., 200 milliseconds or longer, or other appropriate time period selected to avoid false positive activation), as described previously herein.

In the depicted embodiment of FIGS. 8-23, the hand activation switch 15 is arranged in the housing 100 on the circuit board 16, and is configured to be activated by an externally mounted button 13 such that the user can press the button 13 to not only start up the indicating system 20, but also to activate the energy source 30 and energize the electrosurgical instrument 10 including the end effector electrodes to cauterize or seal the tissue. Alternatively, or in addition thereto, a foot switch 33 (FIG. 7) can be used to energize the electrosurgical instrument 10.

As discussed previously, the indicating system includes one or more indicators for providing at least one audible, visual, or tactile indication to the user depending upon which predetermined operating condition is satisfied. For example, in one embodiment, the indicator (e.g., LED 27) emits a signal to the user when the tissue is fully cauterized or sealed. The present disclosure also contemplates the emission of different or multiple percipient signals including audible, visual, and/or tactile. For example, the signal may be a sound, a light, or a vibration. Furthermore, an on-board power supply (e.g., a battery, not shown) located within the housing 100 provides the indicating system 20 with power. By way of example, the power supply is a battery permanently connected to the indicating system 20, and the indicating system is configured to remain inactive until RF current is flowing in the RF current trace 31 adjacent the GMR sensor 21. As in the previously described embodiments, after the indicating system 20 has been powered on for a period of time (e.g. 30 to 120 minutes) the controller (e.g., the microprocessor 26) can be configured to place the indicating system in a standby mode where no power is used until the button 13 is pressed signaling the controller to switch to the ready mode. In the standby mode the indicating system 20 will not utilize power from the battery in excess of the rated discharge rate in air (e.g., <50 picoamps). This assures the battery will have adequate shelf life (e.g., about 5 years).

As discussed previously, As RF energy is applied to the tissue, the tissue impedance is low allowing the current level to be high, as identified as Stage 1 in FIG. 1. As the moisture in the tissue begins to dissipate, the impedance starts to increase and the current flow starts to drop, as identified as Stage 2 in FIG. 1. Once blood is coagulated, tissue is cauterized or the vessel adequately sealed the current remains constant or nearly constant, as identified as Stage 3 in FIG. 1. Stage 3 may occur at any current level (or amperage) dependent upon, for example, the blood is coagulated, tissue is cauterized, or vessel(s) sealed. The current may fluctuate during sealing for various reasons but the indicating system 20 is configured such that the RF current must be stable for a predetermined minimum period of time in order to trigger the activation of a signal to the user. Stage 3 may occur at any current level (or amperage) dependent upon the tissue being sealed or cauterized, but no predetermined current level or tissue impedance is required for detection of Stage 3 or to signal the user.

In some embodiments, a similar profile can be used to detect when an electrical short occurs. For example, since a short will typically result in a steady but higher than normal RF current. The microprocessor 26 of the controller can be programed to distinguish between a steady RF current resulting from adequate tissue sealing or cauterization and a steady RF current resulting from a short. The indicating system 20 in these embodiments is also configured to provide discernably different indications to the user so that the user will know whether there is a short or tissue treatment has been adequately completed.

By way of example, the electrosurgical instrument can include one visual indicator (e.g., an LED of a first color) for indicating adequate tissue treatment and another visual indicator (e.g., a second LED emitting a second color of light) for indicating that a short has been detected. Alternatively, the controller can be configured such that LED 27 changes from a first state to a second state (e.g. goes from off to on) for indicating adequate tissue treatment, and changes to a third state (e.g., blinking on and off) for indicating that a short has been detected. As yet another alternative, two (or more) different types of indicators can be provided such as an LED for indicating adequate tissue treatment, and a buzzer or other type of audible indicator for indicating that a short has been detected.

In another alternative embodiment the controller further includes a timer device or a timer function (e.g., programed into microprocessor 26) for monitoring the elapsed time that RF energy is applied to the tissue such that one or more indicators 23 change state (e.g., are activated) when a predetermined amount of time has passed while energy is applied or a total accumulated time has passed for repeated applications of energy that is sufficient for blood to be coagulated, tissue is cauterized or vessel(s) sealed.

In some instances it may be desirable to selectively continue tissue treatment for an extended period of time beyond when the RF current through the tissue is stable, particularly when treating larger portions of tissue (e.g., vessels having a diameter of about 7 mm or a greater). While the surgeon can simply delay the cessation of RF current once the indicating system has indicated that the RF current is stable in order to provide additional assurance that adequate sealing has been achieved, an alternative embodiment of the indicating system 20 allows the user to selectively delay the activation of the indicator signaling adequate tissue treatment. In this embodiment, an additional input device (e.g., a button or switch) is provided on the housing of the indicating system or on the electrosurgical instrument when the indicating system is provided therein, wherein the additional input device allows the user to select an extended treatment mode. When the extended treatment mode is selected, activation of the indicator signaling adequate tissue treatment is delayed for a predetermined period of time (e.g., 0.5 to 3 seconds, 0.5 to 2 seconds, or 0.5 to 1 second) after the RF current is stable. Particularly when a microprocessor is included in the controller for determining when the RF current is stable, this can be accomplished by programming the microprocessor such that the predetermined period of time that the rate of change of the sensor signal must be substantially zero in order to identify adequate treatment is increased when the extended treatment mode is selected.

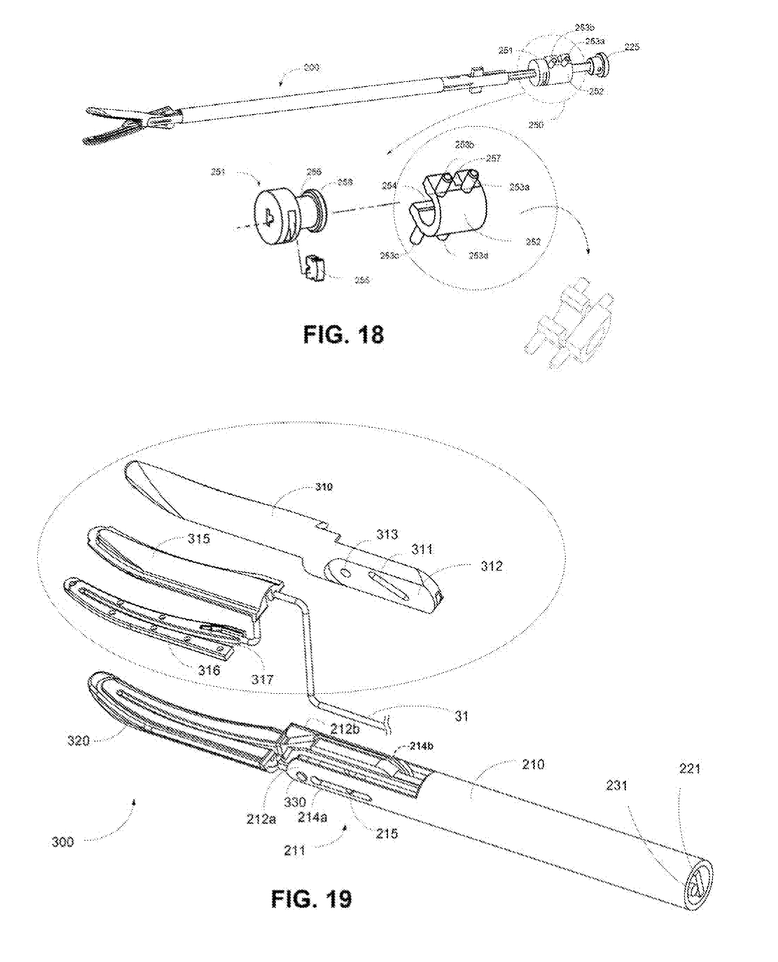

Further referring to FIG. 12, the driving assembly 220 is side-by-side with the knife assembly 230 through connecting assembly 250, which cooperates with the trigger assembly 120 to reciprocate the knife assembly 230 with respect to the jaw driving member 221 of the driving assembly 220, and cooperates with the rotating assembly 130 to rotate the knife assembly 230 together with the jaw driving member 221 of the driving assembly 220 and the shaft 210 of the elongated shaft portion 200. More specifically, referring to FIGS. 12 and 18 the connecting assembly 250 includes a first connector 251 and a second generally U-shaped connector 252 configured to receive the proximal portion of the first connector 251 and connect the trigger assembly. The U-shaped connector 252 is provided four arms 253a-d disposed on both sides thereof. The connecting assembly 250 also includes a locker 255; accordingly, the knife rod 231 of the knife assembly 230 includes an annular groove 234 which cooperates with the locker 255 for securing the first connector 251 with the knife rod 231 of the knife assembly 230. The U-shaped connector 252 defines a cavity 254 for receiving the middle portion 256 of the first connector 251, and a recess 257 receiving the proximal flange 258 of the first connector 251, such that, the knife assembly 230 can be reciprocated but is non-rotatable with respect to the jaw driving member 221 of the driving assembly 220.

As shown in FIGS. 18, and 20-22, the knife trigger 120 includes a trigger portion 124 and two bifurcated ends 122a and 122b disposed on the proximal end thereof. Accordingly, the connector 252 is seated within a cavity defined between the bifurcated ends 122a and 122b. A pair of slots 123a and 123b are disposed on the bifurcated ends 122a and 122b, respectively, which are dimensioned to receive arms 253a and 253d of the second connector 252, respectively. In addition, the distal edge of the bifurcated ends 122a and 122b are contacted against arms 253b and 253c of the connector 252, such that, pivoting the trigger portion 124 of the knife trigger 120 towards the stationary handle 140 about a pivot pin 121 causes the arms 253a-d of the connector 252 to reciprocate, with the arms 253a and 253d of the connector 252 sliding within the slots 123a and 123b, causing the knife assembly 230 to reciprocate.

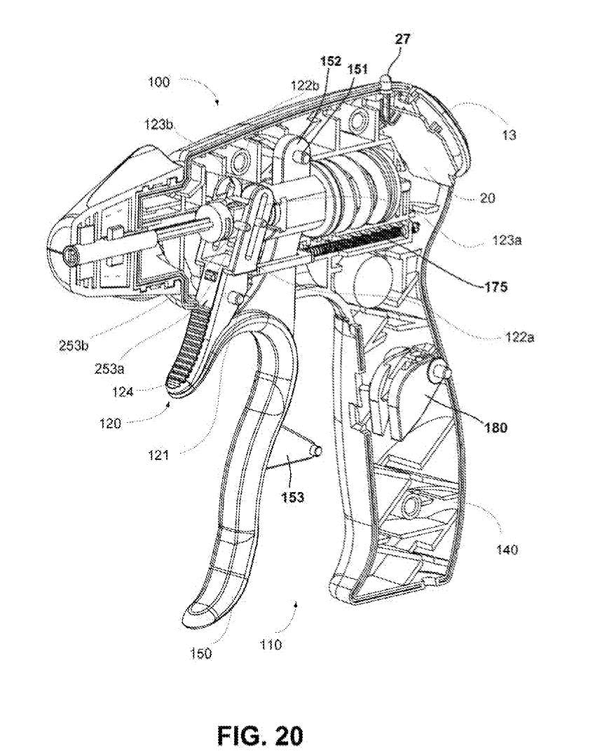

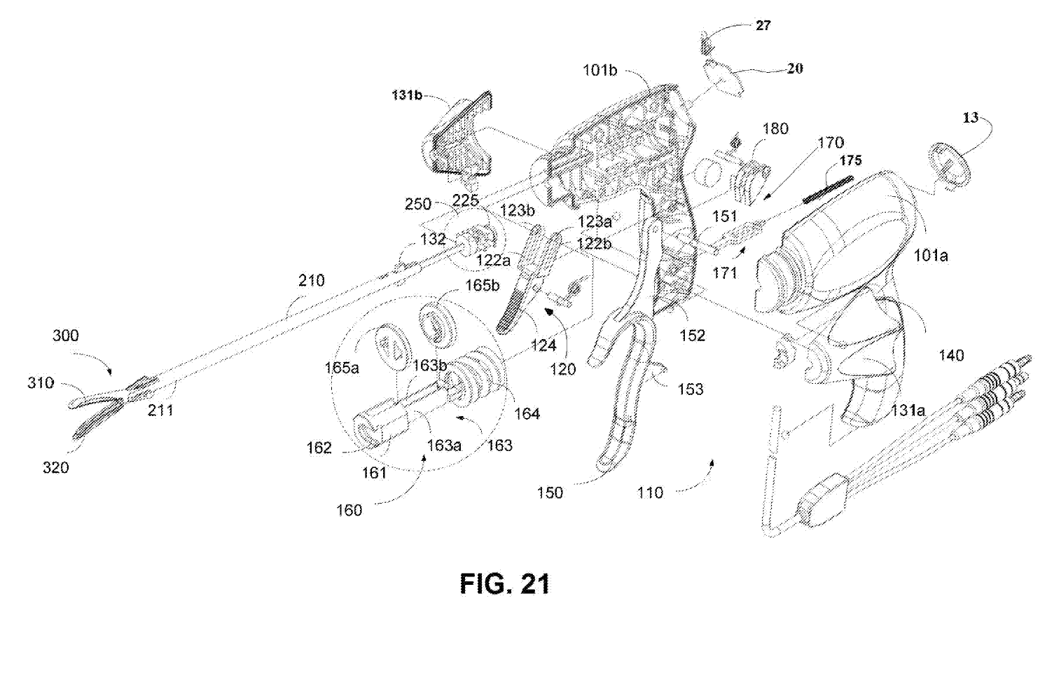

The drive assembly 220 is positioned within the housing 100 between the housing halves 101a and 101b (FIG. 9). As described above, the drive assembly 220 includes the previously described jaw driver 221 and a compression mechanism 160 (FIG. 21). The compression mechanism 160 includes a compression sleeve 161, the distal end of which is provided with a notch 162 for receiving a flange 226 of the tab 225 (FIG. 17) such that longitudinal movement of the compression sleeve 161 actuates the jaw driver 221. The proximal portion of the compression sleeve 161 is provided with a spring mount 163 dimensioned as bifurcated arms 163a and 163b, so as to allow a spring 164 to slide over the bifurcated arms 163a and 163b. More specifically, the compression mechanism 160 also includes a pair of fixing members 165a and 165b, as best shown in FIG. 16, so as to mount the spring 164 on the proximal portion of the compression sleeve 161. The bifurcated arms 163a and 163b define a cavity therebetween, with the arm 152 of the movable handle 150 passing through this cavity so as to be mounted with the pivot pin 151 (see FIG. 20).

The end effector 300 is also rotatable. Thus, the forceps 10 includes a rotating assembly 130 comprising two halves 131a and 131b (see FIG. 21) which, when assembled, enclose and engage the proximal end 212 of the shaft 210 to permit selective rotation of the end effector 300 as needed. As seen in FIG. 17, a fixing member 132 is located within a slot in the proximal end 212 of the shaft 210, with the jaw driver 221 and the knife rod 231 extending through the fixing member 132. The fixing member 132 includes two wings such that, when mounted in the rotating assembly halves 131a and 131b, the fixing member 132 rotates when the rotating assembly 130 is rotated, thereby causing the shaft 210 and the jaw closing assembly 220 to rotate within the insulating tube 260 so as to rotate the end effector 300.

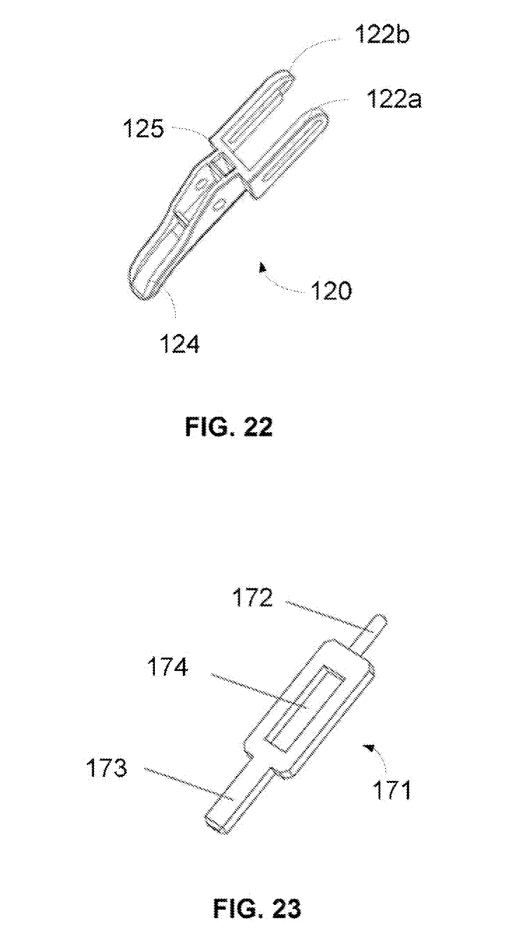

The bipolar forceps 10 also includes a safety mechanism 170 (FIG. 21) configured to prevent actuation of the knife trigger 120 and the knife assembly 230 before closing the end effector 300. More specifically, the safety mechanism 170 includes a recess 125 arranged on the back surface of the trigger portion 124 of the knife trigger 120 (see FIG. 22), and a safety member 171 having two arms 172 and 173 (see FIG. 23). The proximal arm 172 of the safety member 171 is configured to be connected with a spring 175 (see FIG. 21), and the distal arm 173 of the safety member 171 is configured to be inserted into the recess 125 of the trigger member 120 (as shown assembled in FIG. 20). In addition, an aperture 174 is arranged in the middle portion of the safety member 171 that allows the arm 152 of the movable handle 150 to pass through.

As best illustrated in FIGS. 20 and 21, when the movable handle 150 is pivoted towards the stationary handle 140, the arm 152 of the movable handle 150 forces the safety member 171 to move proximally and biasing the spring 175, such that the distal arm 173 of the safety member 171 is disconnected from the recess 125 of the knife trigger 120, enabling the knife trigger 120 to be actuated.

Further referring to FIG. 21, the bipolar forceps 10 further includes a locking member 180 which cooperates with a cam flange 153 of the movable handle 150 so as to lock the handle in the closed position, and releasing the handle 150 upon further pivoting of the movable handle 150 towards the stationary handle 140. Spring 164 apples forces to the mechanism to compress tissue and allow further movement of the movable handle 150 allowing the locking member 180 to operate with various thickness of tissue.

While various embodiments have been described in detail above, it will be understood that the components, features and configurations, as well as the methods of manufacturing the devices and methods described herein are not limited to the specific embodiments described herein.

What is claimed is:

1. A system for determining the adequacy of electrosurgical treatment of tissue by an electrosurgical instrument that applies RF current to tissue comprising:
(a) a sensor for monitoring the RF current; and
(b) controller circuitry for determining when the RF current flowing through the tissue is stable.

2. The system according to claim 1, wherein the controller circuity determines that the RF current flowing through the tissue is stable by monitoring the rate of change of the RF current.

3. The system according to claim 3, wherein the controller circuity determines that the RF current flowing through the tissue is stable by determining that the rate of change of the RF current is at or below a predetermined level or within a predetermined range.

4. The system according to claim 3, wherein the predetermined level is substantially zero.

5. The system according to claim 2, wherein the sensor is adapted to provide to the controller circuitry a signal indicative of the RF current flowing through the tissue.

6. The system according to claim 5, wherein the sensor provides to the controller circuitry a signal that is proportional to the RF current flowing through the tissue.

7. The system according to claim 3, wherein the controller circuity determines that the RF current flowing through the tissue is stable by determining that the rate of change of the RF current is at or below a predetermined level or within a predetermined range, for a predetermined period of time.

8. The system according to claim 3, wherein the controller circuity determines that the RF current flowing through the tissue is stable by determining that the rate of change in the sensor signal is at or below a predetermined level or within a predetermined range, for a predetermined period of time.

9. The system according to claim 6, wherein the predetermined period of time is at least about 100 milliseconds, at least about 200 milliseconds, at least about 500 milliseconds, or at least about 1000 milliseconds.

10. The system according to any one of claims 1-9, further comprising at least one indicator responsive to the controller circuitry and providing an indication that the electrosurgical treatment is adequate and that the application RF current to the tissue can be discontinued.

11. The system according to claim 10 wherein the indication is at least one of an audible indication, a visual indication and a tactile indication.

12. The system according to any one of claims 1-9, wherein the sensor comprises a GMR sensor.

13. The system according to claim 10, wherein the sensor comprises a GMR sensor for indirectly monitoring the RF current through tissue such that the GMR sensor is located in the magnetic field adjacent a conductor through which the RF current is conducted, but the GMR sensor is not in electrical communication with the RF current or any conductor through which the RF current is conducted to or from the tissue.

14. The system according to any one of claims 1-9, wherein the controller circuitry includes a microprocessor adapted to determine when the RF current flowing through the tissue is stable.

15. The system according to claim 12, wherein the controller circuitry includes a microprocessor adapted to determine when the RF current flowing through the tissue is stable based on a signal provided by the GMR sensor.

16. The system according to any one of claims 1-9, further comprising a housing, wherein the controller circuitry is located within said housing.

17. The system according to claim 12, further comprising a housing, wherein the controller circuitry is located within said housing.

18. The system according to claim 16, wherein said housing is adapted to be operatively located between the electrosurgical instrument and a generator for supplying RF current to the electrosurgical instrument such that the system provides electrical communication between the electrosurgical instrument and the generator.

19. The system according to claim 17, wherein said housing is adapted to be operatively connected to the electrosurgical instrument by at least one instrument cable and to the generator by at least one generator cable.

20. The system according to any one of claims 1-9, further comprising:
a housing, wherein the controller circuitry is located within the housing;
wherein the sensor is adapted to provide to said controller circuitry a signal indicative of the RF current flowing through the tissue, and further wherein said housing is adapted to be located between the electrosurgical instrument and a generator for supplying RF current to the electrosurgical instrument such that the system provides electrical communication between the electrosurgical instrument and the generator.

21. The system according to claim 20, wherein said housing is adapted to be operatively connected to the electrosurgical instrument by at least one instrument cable and to the generator by at least one generator cable such that the system provides electrical communication between the electrosurgical instrument and the generator.

22. The system according to claim 21, wherein the system includes at least two electrical pathways for conducting RF current between an electrosurgical instrument and generator operatively connected to the system, wherein the sensor is electrically isolated from said at least two electrical pathways.

23. The system according to claim 22, wherein the sensor comprises a GMR sensor located to be within the magnetic field that surrounds one of said electrical pathways when RF current is transmitted therethrough.

24. The system according to any one of claims 20-23, further comprising a power supply located within said housing.

25. The system according to any one of claims 1-9, further comprising a housing and a power supply, wherein the controller circuitry and the power supply are located within said housing.

26. The system according to claim 25, wherein said housing is adapted to be located between the electrosurgical instrument and a generator for supplying RF current to the electrosurgical instrument such that the system provides electrical communication between the electrosurgical instrument and the generator.

27. The system according to any one of claims 1-9, further comprising an electrosurgical instrument adapted for applying RF current to tissue.

28. The system according to claim 27, wherein said electrosurgical instrument includes first and second opposed jaw members adapted for clamping therebetween tissue to be treated.

29. The system according to claim 28, wherein said electrosurgical instrument comprises bipolar forceps.

30. The system according to claim 27, further comprising at least one indicator responsive to the controller circuitry and providing an indication that the electrosurgical treatment is adequate and that the application RF current to the tissue can be discontinued.

31. The system according to claim 30 wherein the indication is at least one of an audible indication, a visual indication and a tactile indication.

32. The system according to claim 27, wherein the sensor comprises a GMR sensor.

33. The system according to claim 29, wherein the sensor comprises a GMR sensor.

34. The system according to claim 32, wherein the controller circuitry includes a microprocessor adapted to determine when the RF current flowing through the tissue is stable based on a signal provided by the GMR sensor.

35. The system according to claim 32, wherein the controller circuitry and GMR sensor are provided in the electrosurgical instrument.

36. The system according to claim 35, wherein the system includes an electrical pathway for conducting RF current to an end effector of the instrument, wherein the GMR sensor is electrically isolated from said electrical pathway.

37. The system according to claim 36, wherein the GMR sensor is located to be within the magnetic field that surrounds said electrical pathway when RF current is transmitted therethrough.

38. The system according to any one of claims 10-26, further comprising an electrosurgical instrument adapted for applying RF current to tissue.

39. The system according to claim 38, wherein said electrosurgical instrument includes first and second opposed jaw members adapted for clamping therebetween tissue to be treated.

40. The system according to claim 38, further comprising at least one indicator responsive to the controller circuitry and providing an indication that the electrosurgical treatment is adequate and that the application RF current to the tissue can be discontinued.

41. The system according to claim 39 wherein the indication is at least one of an audible indication, a visual indication and a tactile indication.

42. The system according to any one of claims 38-41, wherein the sensor comprises a GMR sensor.

43. The system according to any one of claims 38-42, wherein the controller circuitry and GMR sensor are provided in the electrosurgical instrument.

44. The system according to claim 43, wherein the system includes an electrical pathway for conducting RF current to an end effector of the instrument, wherein the GMR sensor is electrically isolated from said electrical pathway.

45. The system of any one of claims 1-15, further comprising an electrosurgical generator adapted for supplying RF current to an electrosurgical instrument.

46. The system according to claim 45, further comprising at least one indicator responsive to the controller circuitry and providing an indication that the electrosurgical treatment is adequate and that the application RF current to the tissue can be discontinued.

47. The system according to claim 46 wherein the indication is at least one of an audible indication, a visual indication and a tactile indication.

48. The system according to claim 45, wherein the sensor comprises a GMR sensor.

49. The system according to claim 48, wherein the controller circuitry and GMR sensor are provided in the electrosurgical generator.

50. The system according to claim 49, wherein the system includes an electrical pathway for conducting RF current to an electrosurgical instrument operatively connected to the generator, wherein the GMR sensor is electrically isolated from said electrical pathway.

51. The system according to claim 50, wherein the GMR sensor is located to be within the magnetic field that surrounds said electrical pathway when RF current is transmitted therethrough.

52. An indicating system for signaling the adequacy of an electrosurgical treatment of tissue by an electrosurgical instrument applying RF current to the tissue, the indicating system comprising:
(a) a sensor for monitoring the RF current applied to the tissue and providing a signal indicative of the RF current flowing through the tissue;
(b) at least one indicator for providing an indication to an operator of the electrosurgical instrument that the electrosurgical treatment is adequate so that application of RF current to the tissue can be discontinued; and
(b) a controller configured to determine if the RF current flowing through the tissue is stable based on the signal provided by the sensor;
wherein the state of the at least one indicator is changed when the RF current flowing through the tissue is stable.

53. The indicating system according to claim 52, wherein the sensor provides to the controller circuitry a signal that is proportional to the RF current flowing through the tissue.

54. The indicating system according to claim 53, wherein the controller determines that the RF current flowing through the tissue is stable by determining that the rate of change in the sensor signal is at or below a predetermined level.

55. The indicating system according to claim 54, wherein the predetermined level is substantially zero.

56. The indicating system according to claim 52 wherein the indication is at least one of an audible indication, a visual indication and a tactile indication.

57. The indicating system according to any one of claims 51-56, wherein the sensor comprises a GMR sensor.

58. The indicating system according to claim 57, wherein the controller includes a microprocessor adapted to determine if the RF current flowing through the tissue is stable based on the signal provided by the GMR sensor.

59. The indicating system according to claim 57, wherein the system includes an electrical pathway for conducting RF current between the electrosurgical instrument and a generator operatively connected to the system, wherein the GMR sensor is located to be within the magnetic field that surrounds said electrical pathway when RF current is transmitted therethrough, and further wherein the GMR sensor is electrically isolated from said electrical pathway.

60. An electrosurgical instrument adapted for applying RF current to tissue, said electrosurgical instrument including the indicating system of claim 59.

61. The electrosurgical instrument of claim 60, wherein said electrosurgical instrument includes first and second opposed jaw members adapted for clamping therebetween tissue to be treated.

62. The electrosurgical instrument of claim 61, wherein said electrosurgical instrument comprises bipolar forceps.

63. The electrosurgical instrument of claim 62, wherein the controller includes a microprocessor adapted to determine if the RF current flowing through the tissue is stable based on the signal provided by the GMR sensor.

64. An electrosurgical instrument adapted for applying RF current to tissue, wherein the indicating system according to any one of claim 1-15 or 52-59 is integral to or contained within or on the instrument.

65. The electrosurgical instrument of claim 64, wherein the instrument is adapted for at least one of sealing, cutting, ablating and cauterizing tissue.

66. A system for monitoring the electrosurgical treatment of tissue by an electrosurgical instrument that applies RF current to tissue comprising:
(a) a sensor for indirectly monitoring the RF current flowing through the tissue and generating a signal proportional to the magnitude of said RF current;
(b) a controller adapted to determine at least one state of tissue treatment based on the signal from said sensor; and
(c) at least one indicator responsive to the controller;
wherein the controller changes the state of the at least one indicator based on a determined state of tissue treatment.

67. The system of claim 66, wherein the controller determines when the application of RF current to tissue commences, and the state of tissue treatment is at least one of: the period of time elapsed since RF current commenced; the total period of time that RF current has flowed through the tissue; the period of time elapsed since the RF current reached a maximum magnitude; and the period of time elapsed since the rate of change of the RF current met a predetermined condition.

68. The system of claim 66, wherein the controller changes the state of the at least one indicator when the period of time meets or exceeds a predetermined amount.

69. The system of claim 66, wherein the controller determines when the rate of change of the RF current is within a predetermined range.

70. A system for detecting an electrical short in an electrosurgical system comprising:
a sensor to generate a signal that is relative to the electrosurgical current flowing between electrodes; and
circuitry for determining when the current is stable and is higher than a threshold level typical of adequate electrosurgical sealing or cauterization.

71. The system according to claim 70, wherein the controller circuity determines that the RF current flowing through the tissue is stable by monitoring the rate of change of the RF current.

72. The system according to claim 71, wherein the controller circuity determines that the RF current flowing through the tissue is stable by determining that the rate of change of the RF current is at or below a predetermined level or within a predetermined range.

73. The system of claim 72 where an electrical short is detected when the rate of change of the RF current is at or below a predetermined level or within a predetermined range for a predetermined period of time.

74. The system of any one of claims 70-73 configured to communicate the detection of a short to a controller or to the user to switch off the flow of electrosurgical energy or correct the short.

75. The system of any preceding claim wherein one or more components of the system is powered by a battery, capacitor, photovoltaic array, an induction coil or other methods to store power or to harvest power from the electrosurgical energy used in the treatment.

76. A method for determining the adequacy of treatment for sealing, cutting or cauterization tissue when using a electrosurgical device comprising:
detecting when current is being applied to the tissue;
determining when a predetermined time that energy is applied to tissue has elapsed; and
signaling the user when the elapsed time exceeds a predetermined length time.

77. The method according to claim 76 wherein the length of time is cumulative or is the total of two more applications of the energy through the tissue.

78. The method according to claim 76 or 77, wherein:
the length of time that energy is applied through the tissue added on to the time required for the current to stabilize.

79. The method according to claim 76 or 77 wherein the predetermined time is adequate for sealing, cutting or cauterization tissue.

80. The method according to claim 76 or 77 further comprising the step of controlling the flow of electrosurgical energy through the tissue to control electrical parameters of the energy or to switch it off.

81. The method of claim 76 or 77 wherein the current is sensed by a GMR sensor.

82. A method for determining the adequacy of tissue sealing, cauterization or cutting comprising the steps of:
monitoring electrosurgical current being applied to tissue;
determining when the detected current is stable or when the current has been applied to the tissue in excess of a predetermined length of time; and
signaling the user to manually discontinue the flow of current through the tissue or automatically controlling or discontinuing the flow of current through the tissue.

83. The method of claim 82 wherein determining when the detected current is stable comprises determining when the first time derivative of the current is within a predetermined level.

84. The method of claim 82 or 83 wherein determining when the detected current is stable comprises determining when the first time derivative of the current is within a predetermined range, for a predetermined period of time.

85. The method of claim 82 where the predetermined length of time is adequate to seal or cauterize the tissue.

86. The method of any one of claim 82, 83 or 85 wherein the current is sensed by a GMR sensor.
